(19) United States
(12) Patent Application Publication
(10) Pub. No.: US 2025/0161429 A1
(43) Pub. Date: May 22, 2025

(54) PAN-PNEUMOVIRUS VACCINE COMPOSITIONS AND METHODS OF USE THEREOF

(71) Applicant: University of Georgia Research Foundation, Inc., Athens, GA (US)

(72) Inventors: Jarrod Mousa, Athens, GA (US); Jiachen Huang, Athens, GA (US)

(21) Appl. No.: 18/838,554

(22) PCT Filed: Feb. 14, 2023
(86) PCT No.: PCT/US2023/062591

§ 371 Date: Aug. 14, 2024

Related U.S. Application Data
(60) Provisional application No. 63/309,973, filed on Feb. 14, 2022.

(51) Int. Cl. A61K 39/12; A61K 39/00; A61P 37/04; C07K 14/005; C12N 15/86

(52) U.S. Cl. CPC A61K 39/12; A61P 37/04; C07K 14/005; C12N 15/86; A61K 2039/55; C07K 2319/02; C07K 2319/40; C12N 2760/18322; C12N 2760/18334; C12N 2760/18343; C12N 2760/18522; C12N 2760/18534; C12N 2760/18543

(57) ABSTRACT

Chimeric polypeptides, chimeric fusion proteins, immunogenic compositions, and methods of use thereof for pan-pneumovirus vaccination are provided. The chimeric polypeptides typically include immunodominant epitopes of the fusion protein of respiratory syncytial virus (RSV) and human metapneumovirus (hMPV), and preferably include one or more of antigenic sites Ø, V, and II of RSV, and III, IV, and DS7 of hMPV. The chimeric polypeptides can be utilized as the antigenic domain in chimeric fusion proteins include one or more additional domains such as a signal peptide sequence, a trimerization domain, a cleavage site, a purification tag or report sequence, and one or more linker sequences. Nucleic acids encoding the chimeric polypeptides and chimeric fusion proteins are also provided, as are recombinant viruses having the same. Pharmaceutical compositions including one or more of the foregoing compositions, and methods of there use for immunizing subjects against RSV and hMPV are also provided.

### CROSS-REFERENCE TO RELATED APPLICATIONS

This application claims the benefit of and priority to U.S. Ser. No. 63/309,973 filed Feb. 14, 2022, and which is incorporated by reference herein in its entirety.

### STATEMENT REGARDING FEDERALLY SPONSORED RESEARCH OR DEVELOPMENT

This invention was made with government support under R01AI143865 awarded by the NIH. The government has certain rights in the invention.

### REFERENCE TO THE SEQUENCE LISTING

The Sequence Listing submitted as a text file named “UGA_2022-094-02_PCT_ST26.xml” created on Feb. 14, 2023, and having a size of 46,130 bytes is hereby incorporated by reference pursuant to 37 C.F.R. § 1.834(c)(1).

### FIELD OF THE INVENTION

The field of the invention generally relates to immunogenic compositions and methods of use thereof to increase immune responses against pneumoviruses.

### BACKGROUND OF THE INVENTION

Respiratory syncytial virus (RSV) and human metapneumovirus (hMPV) are significant causes of acute lower respiratory tract infections (ALRI) in infants and young children (Nair et al., Lancet, 375:1545-1555 (2010), Shi et al., Lancet 390:946-958 (2017), Edwards et al., N Engl J Med 368:633-643 (2013), Schuster & Williams, Pediatr Rev 34:558 (2013)). RSV was first identified in 1956, and was subsequently recognized as a common cause of respiratory illness in early life (Chanock et al., Am J Hyg 66:291-300 (1957)). The majority of children experience at least one RSV infection before 2 years of age, and infants under 6 months old have a higher risk of severe disease requiring hospitalization (Hall et al., N Engl J Med 360:588-598 (2009)). hMPV was identified in 2001, and it has been found to be the second most common cause of viral lower respiratory infection in children (den Hoogen et al., Nat Med 7:719-724 (2001)). In contrast to RSV, the peak age for infant hospitalizations caused by hMPV infections is 6-12 months old, with a nearly 100% exposure rate by the age of 5 (Edwards et al., N Engl J Med 368:633-643 (2013)). Reinfections of both RSV and hMPV are common throughout life, which usually cause mild symptoms in healthy adults. However, for certain populations including immunocompromised patients, individuals over 65 years of age, and people with underlying conditions such asthma or chronic obstructive pulmonary disease (COPD), infection with RSV or hMPV may lead to severe bronchiolitis and pneumonia (Falsey et al., N Engl J Med 352:1749-1759 (2005), Falsey et al., J Infect Dis 209:1873-1881 (2014), Dowell et al., J Infect Dis 174:456-462 (1996), Panda et al., Int J Infect Dis 25:45-52 (2014)).

The fusion (F) glycoproteins of RSV and hMPV are highly similar in structure and share ˜30% amino acid sequence identity. Both F proteins belong to the class I viral fusion protein family and play indispensable roles in viral attachment as well as membrane fusion. To become fusion competent, the F0 precursor must be cleaved into F1 and F2 subunits that are linked by two disulfide bonds to generate a mature meta-stable homotrimer (Jardetzky & Lamb, Curr Opin Virol 5:24-33 (2014)). RSV F is cleaved at two furin cleavage sites with the p27 fragment in between F1 and F2 removed, whereas hMPV F has only one cleavage site that can be cleaved by the host membrane protease TMPRSS2 (Shirogane et al., J Virol 82:8942-8946 (2008)). To initiate the fusion process, the hydrophobic fusion peptide on the N terminus of the F2 subunit is exposed and inserted into host cell membrane, which triggers the conformational rearrangements that turns the F protein into the stable post-fusion state, and brings the viral and host cell membranes together for lipid mixing.

Multiple antigenic sites have been identified on both RSV F and hMPV F proteins. Among the six known antigenic sites of RSV F, pre-fusion-specific sites Ø and V are targeted by over 60% of neutralizing antibodies in humans (Ngwuta et al., Sci Transl Med 7:309ra162-309ra162 (2015), Gilman et al., Sci Immunol 1 (2016)), indicating these sites are vital for immune recognition and antibody neutralization (Andreano et al., EMBO Mol Med e14035 (2021)). hMPV F shares three antigenic sites (III, IV, V) with RSV F, as several antibodies have been found to cross react with RSV and hMPV F at these epitopes (Más et al., PLoS Pathog 12:e1005859 (2016), Wen et al., Nat Microbiol 2:1-7 (2017), Mousa et al., PLoS Pathog 14:e1006837 (2018), Xiao et al., In MAbs (2019)). In addition, the area in between site III and IV was found to be a distinct hMPV site that is recognized by a mAb called DS7 (Williams et al., J Virol 81:8315-8324 (2007), Wen et al., Nat Struct & Mol Biol 19:461 (2012)). Studies have shown that, unlike RSV F-specific antibodies, the majority of hMPV F-specific antibodies target epitopes present in both pre-fusion and post-fusion conformations, likely due to glycosylation present near pre-fusion-specific sites on the head of hMPV F (Pilaev et al., Vaccine 38:2122-2127 (2020), Battles et al., Nat Commun 8:1-11 (2017)).

Currently, there are no vaccines available for either RSV or hMPV. Previous attempts with formalin-inactivated RSV and hMPV vaccines revealed that low affinity, non-neutralizing F-specific antibodies induced by denatured fusion proteins cannot provide protection, and lead to vaccine enhanced disease (Killikelly et al., Sci Rep 6:1-7 (2016), Murphy et al., J Clin Microbiol 24:197-202 (1986), Murphy & Walsh, J Clin Microbiol 26:1595-1597 (1988), de Swart R L, et al., Vaccine 25:8518-8528 (2007), Yim et al., Vaccine 25:5034-5040 (2007)). By stabilizing the F proteins in the pre-fusion conformation, several studies have demonstrated improvement in neutralizing antibody titers (McLellan et al., Science (80-) 342:592-598 (2013), Huang et al., J Virol JVI0059321), while for hMPV, pre-fusion and post-fusion F proteins induced comparable neutralizing antibodies in mice (Battles et al., Nat Commun 8:1-11 (2017)) and immunization with post-fusion F completely protected mice from hMPV challenge (Huang et al., J Virol JVI0059321). Several epitope-focused vaccine designs have been tested for RSV and hMPV F. A head-only RSV F protein boosted titers of neutralizing Abs targeting antigenic sites Ø and II (Boyington et al., PLoS One 11:e0159709 (2016)). In a different study, RSV F was modified by glycan-masking that blocked poorly neutralizing epitopes on a nanoparticle, which induced a more potent neutralizing Ab response than a prefusion F trimer (Swanson et al., Sci Immunol 5:eaba6466 (2020)). Based on computational protein design strategies, RSV F site II was presented on a scaffold fused with RSV N-based nanoparticles, which boosted subdominant neutralizing antibody responses targeting antigenic site II in mice (Sesterhenn et al., PLoS Biol 17:e3000164 (2019), Correia et al., Nature 507:201-206 (2014)). In addition, RSV F neutralizing antigenic sites (Ø, II, IV) were tested on de novo protein scaffolds respectively, and a mixture of these epitope-based immunogens induced focused immune responses toward the target antigenic sites (Sesterhenn et al., Science (80-) 368 (2020)). All of the studies above demonstrate the concept that engineered RSV F epitope-based immunogens can induce and boost neutralizing RSV F antibodies.

The idea of universal vaccine development provides the possibility of preventing multiple variants (like flu and HIV) by a single immunogen. Due to the similarities between RSV and hMPV F, researchers have tried to generate universal RSV/MPV vaccines by grafting the helix-turn-helix motif (site II) from RSV F onto hMPV F, however, this chimera induced neutralizing antibody responses only to hMPV, but not RSV (Wen et al., PLoS One 11:e0155917 (2016)). A similar study that grafted RSV F and hMPV F epitopes on pre-fusion and post-fusion F proteins showed that chimeric proteins swapping either site II or site IV can induce cross-neutralizing antibodies in mice, but a challenge with pre-fusion candidates was lacking (Olmedillas et al., EMBO Mol Med 10:175-187 (2018)). For the influenza hemagglutinin (HA) protein, chimeric immunogens generated by swapping the HA head with zoonotic subtypes while retaining the conserved HA stem successfully induced antibodies targeting the subdominant HA stem (Nachbagauer et al., npj Vaccines 1:1-10 (2016), Nachbagauer et al., Nat Med 27:106-114 (2021)).

In sum, multiple promising vaccine designs based on the fusion proteins of RSV and hMPV have been tested, but none of them can induce cross-protective antibody responses despite their similar structures and closely related amino acid sequence identity.

Therefore, there remains a need for improved vaccine technology against RSV and hMPV.

It is an object of the invention to provide alternative pan-pneumovirus immunogens for inducing immune responses against RSV and hMPV

### SUMMARY OF THE INVENTION

Chimeric immunogens that contain elements from the head of RSV F and the stem of hMPV F, and compositions containing the same, and methods of use thereof are provided. The chimeric proteins typically combine the immunodominant epitopes of the RSV and hMPV fusion proteins into a single antigen (also referred to herein as bivalent antigen or vaccine). Preferably the chimeric proteins maintain the immunological features of both RSV F and hMPV F that can be recognized by epitope-specific mAbs and human B cells pre-exposed to RSV or hMPV.

Thus, chimeric polypeptides are provided. The chimeric polypeptides typically include one or more immunodominant epitopes of the fusion protein (F) of respiratory syncytial virus (RSV) and one or more immunodominant epitopes of the fusion protein (F) of human metapneumovirus (hMPV). Typically, the immunodominant epitopes from RSV are derived from the head of RSV F, the immunodominant epitope(s) from hMPV are derived from the stem of hMPV F, or a combination thereof. In preferred embodiments, the chimeric polypeptide includes amino acid sequences of two or more, optionally all, antigenic sites selected from the group consisting of RSV site Ø, RSV site V, RSV site II, hMPV site III, hMPV site IV, and hMPV DS7 site. In some embodiments, the chimeric polypeptide includes two cleavage sites of RSV; one or more DsCav1 mutations (S155C, S190F, V207L, and/or S290C with reference to SEQ ID NO:3); the fusion peptide of RSV F having the F2 N-terminus (residues 26-54 relative to SEQ ID NO:3) and the F1 C-terminus (residues 315-531 relative to SEQ ID NO:3) replaced by the homologous hMPV F sequences, with two junctions located on β2 and β7 strands; two glycosylation sites (RSV F-N70 relative to SEQ ID NO:3, hMPV F-N354 relative to SEQ ID NO:4 or hMPV F-N353 relative to SEQ ID NO:40); or a combination thereof. Specific chimeric polypeptide sequences are provided and can include, for example, the amino acid sequence of SEQ ID NO:1, a fragment thereof, or a variant thereof optionally having at least 50, 60, 70, 75, 80, 85, 90, 95, or more percent sequence identity to SEQ ID NO:1.

The chimeric polypeptides can be utilized as the antigenic domain in chimeric fusion proteins including one or more additional domains. Such additional domains include, but are not limited to, a signal peptide sequence, a trimerization domain, a cleavage site, a purification tag or reporter sequence, and one or more linker sequences. Exemplary signal peptide sequences, trimerization domains, cleavage site sequences, purification tag or reporter sequences, and linker sequences are also provided. For example, in some embodiments, the trimerization domain is selected from a GCN4-based isoleucine zipper (IZ) domain, a T4 bacteriophage fibritin foldon (Fd) trimerization domain, the trimerization domain of collagen, the HIV gp41 trimerization domain, and the transmembrane domain and cytoplasmic tail of RSV F or hMPV F. Optionally, the trimerization domain includes the amino acid sequence of any one of SEQ ID NOS:19-26, 44, or 45. In some embodiments, the peptide signal sequence is derived from RSV F or hMPV F. Optionally, the signal peptide sequence includes the amino acid sequence of SEQ ID NOS:5 or 6. Purification tags can assist with isolation of recombinantly expressed protein and can be, for example, six consecutive histidine residues. In particular embodiments, the domains, if present, are in the orientation from N-terminus to C-terminus: signal peptide sequence-antigenic domain-trimerization domain-cleavage site-purification tag, optionally wherein one or more pairs of domains are separated by a linker sequence.

Specific chimeric fusion proteins are exemplified and can have the amino acid sequence of any one of SEQ ID NOS:2, or 41-43, or 46-47, or fragment, or variant thereof with at least 70% sequence identity thereto.

Nucleic acids encoding the chimeric polypeptides and chimeric fusion proteins are also provided, and can be, e.g., single stranded or double stranded, linear or circular, DNA or RNA, and in the sense or antisense orientation. The encoding nucleic acid sequence operably linked to an expression control sequence, for example in vector such as a plasmid or viral vector, are also provided. The nucleic acids in recombinant viral genomes and antigenomes are also provided. Viruses having incorporating the chimeric polypeptide and/or chimeric fusion protein and optionally having the polypeptide and/or fusion protein encoded by its genome are also provided. The viruses can be, for example, attenuated or unattenuated recombinant RSV or hMPV.

Methods of making of chimeric polypeptides and fusion proteins are also provided and can include expressing a nucleic acid encoding the chimeric polypeptide or fusion protein in cells, optionally human or insect cells, and isolating the expressed chimeric polypeptide or fusion protein. The expressed protein can undergo maturation and/or post-translational modification, e.g., cleavage of a signal sequence if-present, cleavage at one or more internal cleavage site, and/or multimerization (e.g., trimerization) in the cells prior to isolation and/or after isolation. In some embodiments, the chimeric polypeptide or fusion protein is further processed during or after isolation, e.g., to remove a purification tag, etc. Thus, isolated macromolecules formed by multimerization, e.g., trimerization, of the disclosed chimeric polypeptides and fusion proteins are also provided.

Pharmaceutical compositions including one or more of (i) chimeric polypeptide, (ii) chimeric fusion protein, (iii) a nucleic acid, vector, or viral genome or antigenome encoding chimeric polypeptide and/or fusion protein, (iv) recombinant virus incorporating chimeric polypeptide and/or fusion protein, and optional encoding chimeric polypeptide and/or fusion protein in its genome, (v) expressed chimeric polypeptide and/or fusion made according to the disclosed methods, and/or (vi) isolated macromolecules formed by multimerization, e.g., trimerization, of the disclosed chimeric polypeptides and fusion proteins are also provided. In some embodiments, the composition further includes an adjuvant. Thus, immunogenic and vaccine compositions including the chimeric polypeptide and/or fusion protein are provided. Any of the compositions can be packaged in a delivery vehicle such as polymeric or liposomal or protein nanoparticles.

Methods of inducing or increasing an immune response in a subject in need thereof are also provided. The methods typically include administering the subject an effective amount of the composition to induce or increase an immune response, preferably against the chimeric polypeptide (i.e., the antigenic domain of the chimeric fusion protein). In preferred embodiments, the composition increases immunity against one or more pneumoviruses in the subject. The one or more pneumoviruses can include RSV, hMPV, or preferably both. In some embodiments, increased immunity includes an increase in neutralizing antibodies against the one or more pneumoviruses. Methods of administration include, but are not limited to, intranasal and intramuscular delivery.

In some embodiments, the composition is administered prophylactically. Administration can, for example, reduce viral infection and/or one or more symptoms caused by viral infection in the subject. The subject can be a human, for example, an infant, child, or adult optionally elderly adult.

### BRIEF DESCRIPTION OF THE DRAWINGS

FIGS. 1A-1C illustrate RHMS-1 protein design. FIG. 1A is a diagram generated with the head of pre-fusion RSV F and the stem of pre-fusion hMPV F (5WB0) shows one protomer in cartoon and the rest of two protomers in surface. FIG. 1B is a sequence alignment of RSV-A F (SEQ ID NO:3), RHMS-1 (SEQ ID NO:2), and hMPV-A1 F (SEQ ID NO:4) generated by Jalview. The sequences of known antigenic sites are highlighted in gray scale: RSV site Ø, RSV site V, RSV site II, hMPV site III, hMPV site IV, hMPV DS7 site. In RHMS-1 sequence, four Ds-Cav1 mutations are circled in boxes, two N-linked glycosylation sites are underlined and the GCN4 trimerization domain is circled in a dashed box. FIG. 1C is a diagram of RHMS-1 with the antigenic sites colored in accordance with the sequences highlighted in FIG. 1B labelled and displayed. Both FIGS. 1A and 1C were made by ChimeraX. FIG. 1D is the annotated sequence map (SEQ ID NO:3) of wildtype RSV F protein, showing the signal peptide sequence, F2 domain including the head stem regions thereof, cleavage sites, p27 domain, transmembrane domain, and cytoplasmic domains. FIG. 1E is the annotated sequence map (SEQ ID NO:40) of wildtype hMPV F protein, showing the signal peptide sequence, F1 and F2 domains including the head stem regions thereof, cleavage site, transmembrane domain, and cytoplasmic domains.

FIGS. 2A-2C illustrate purification and negative-stain EM of RHMS-1. FIG. 2A is a plot showing size exclusion chromatography curves of RHMS-1, RSV A2 F DsCav1, and trypsinized hMPV B2 F. FIG. 2B is an image of an SDS-PAGE of F proteins in non-reducing and reduced/heated conditions. FIG. 2C is a representative negative-stain electron micrograph of RHMS-1 obtained from fractions 50-60 mL from the size exclusion chromatogram shown in (2A), scale bar: 100 nm.

FIGS. 3A-3D illustrate the antigenic site-specific mAbs binding to F proteins. FIGS. 3A-3C are a series of ELISA binding curves of mAbs targeting different RSV/hMPV F antigenic sites against RHMS-1 (3A), RSV A2 F DsCav1 (3B), and trypsinized hMPV B2 F monomer (3C). FIG. 3D is a heat map chart showing the EC50 values of the binding curves in (3A-3C). The binding curves and the EC50 values were generated by GraphPad Prism.

FIGS. 4A-4B illustrate the serology of human plasma against F proteins. FIGS. 4A and 4B are plots of an area under the curve analysis of plasma IgG binding to RHMS-1 vs. RSV A2 F DsCav1 (4A) and RHMS-1 vs. trypsinized hMPV B2 F monomer (4B). Each dot represents one subject, and the lines indicate the linear regression fit of the data sets. Figures and data analysis was generated by GraphPad Prism.

FIGS. 5A-5H illustrate human PBMCs binding to F proteins. FIGS. 5A-5H are plots showing ELISA OD₄₀₅ nm values of B cell culture supernatants from four subjects binding to RHMS-1 vs. RSV A2 F DsCav1 (5A-5D) and RHMS-1 vs. trypsinized hMPV B2 F monomer (5E-5H). Each dot represents the B cell supernatant in a single well of a 384 well plate initially containing 20,000 PBMCs. Figures were generated by GraphPad Prism.

FIGS. 6A-6F illustrate mouse immunization and challenge studies. FIG. 6A is a diagram and timeline of an immunization study regimen. FIGS. 6B and 6C are plots showing Day 42 serum IgG titers against RSV A2 F DsCav1 (6B), and against trypsinized hMPV B2 F monomer (6C) before challenge. FIGS. 6D and 6E are plots showing serum neutralization against RSV A2 (6D) and B1 strains at 1/50 dilution and against (6E) hMPV CAN/97-83 and TN/93-32 at a 1/150 dilution. FIG. 6F is a plot showing lung viral titers for RSV A2 and hMPV TN/93-32 in the lung homogenates of mice 5 days post-challenge. LOD: limit of detection.

FIG. 7 is a bar graph of the RSV A/A2 and hMPV viral titers in the lungs of cotton rats. RSV A/A2 load in the lungs of cotton rats in Groups A-E was evaluated 5 days after intranasal RSV challenge. RSV-infected animals mock-treated with PBS i.m. (Group A) showed a titer of 5.43 Log₁₀ PFU/g virus in the lungs. hMPV load in the lungs of cotton rats in Groups F-J was evaluated 5 days after intranasal hMPV challenge. hMPV-infected animals treated with PBS i.m. (Group F) showed a titer of 4.81 Log₁₀ PFU/g in the lungs.

FIG. 8 is a bar graph of the RSV A/A2 and hMPV viral titers in the noses of cotton rats. RSV A/A2 load in the noses of cotton rats in Groups A-E was evaluated 5 days after intranasal RSV challenge. hMPV load in the noses of cotton rats in Groups F-J was evaluated 5 days after intranasal hMPV challenge.

FIG. 9 is a bar graph showing the lung histopathology in cotton rats. Pulmonary histopathology was evaluated in all animals 5 days after viral challenge.

FIGS. 10A and 10B are bar graphs showing the RSV neutralizing antibodies and hMPV neutralizing antibodies in cotton rats. Serum neutralizing antibodies against RSV A/A2 (FIG. 10A) and against hMPV (FIG. 10B) were measured in animals immunized with BiVac, infected with RSV or hMPV, immunized with formalin-inactivated RSV (FI-RSV) or hMPV (FI-hMPV), and control naïve animals.

FIGS. 11A and 11B are bar graphs showing the RSV F protein IgG and hMPV IgG antibodies in cotton rats. Serum binding IgG against RSV A/A2 F (FIG. 11A) protein and against hMPV (FIG. 11B) were measured in animals immunized with BiVac, infected with RSV or hMPV, immunized with FI-RSV or FI-hMPV, and control naïve animals.

FIGS. 12A-12D are bar graphs showing the expression of mRNA for RSV NS1 and hMPV L protein (FIG. 12A), and IFN-γ (FIG. 12B), IL-4 (FIG. 12C) and IL-5 (FIG. 12D) as was evaluated in lung samples collected on day 5 via qPCR. Total RNA was extracted from homogenized tissue or cells using the RNeasy purification kit (QIAGEN). The standard curves were developed using serially diluted cDNA sample most enriched in the transcript of interest (e.g., lungs from post-RSV or -hMPV infection of FI-RSV- or FI-hMPV-immunized animals, respectively). The Ct values were plotted against log₁₀ cDNA dilution factor. These curves were used to convert the Ct values obtained for different samples to relative expression units. These relative expression units were then normalized to the level of β-actin mRNA (“housekeeping gene”) expressed in the corresponding sample. For animal studies, mRNA levels were expressed as the geometric mean±SEM for all animals in a group at a given time.

### DETAILED DESCRIPTION OF THE INVENTION

### I. Definitions

As used herein, “attenuated” refers to procedures that weaken an agent of disease (a pathogen). An attenuated virus is a weakened, less vigorous virus. A vaccine against a viral disease can be made from an attenuated, less virulent strain of the virus, a virus capable of stimulating an immune response and creating immunity but not causing illness or less severe illness. Attenuation can be achieved by chemical treatment of the pathogen, through radiation, or by genetic modification, using methods known to those skilled in the art. Attenuation may result in decreased proliferation, attachment to host cells, or decreased production or strength of toxins.

As used herein, the term “nucleic acid(s)” refers to any nucleic acid containing molecule, including, but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. In accordance with standard nomenclature, nucleic acid sequences are denominated by either a three letter, or single letter code as indicated as follows: adenine (Ade, A), thymine (Thy, T), guanine (Gua, G) cytosine (Cyt, C), uracil (Ura, U).

As used herein, the term “polynucleotide” refers to a chain of nucleotides of any length, regardless of modification (e.g., methylation).

As used herein, the term “gene” refers to a nucleic acid (e.g., DNA or RNA) sequence that comprises coding sequences necessary for the production of a polypeptide, RNA (e.g., including but not limited to, mRNA, tRNA and rRNA) or precursor. The polypeptide, RNA, or precursor can be encoded by a full length coding sequence or by any portion thereof. The term also encompasses the coding region of a structural gene and the sequences located adjacent to the coding region on both the 5′ and 3′ ends for a distance of about 1 kb on either end such that the gene corresponds to the length of the full-length mRNA. The term “gene” encompasses both cDNA and genomic forms of a gene, which may be made of DNA, or RNA. A genomic form or clone of a gene may contain the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “nucleic acid molecule encoding,” refers to the order or sequence of nucleotides along a strand of nucleotides. The order of these nucleotides can determine the order of amino acids along the polypeptide (protein) chain. The nucleotide sequence can thus code for the amino acid sequence.

As used herein, “heterologous” means derived from a different species.

As used herein, a “vector” is a replicon, such as a plasmid, phage, or cosmid, into which another DNA segment may be inserted so as to bring about the replication of the inserted segment. The vectors described herein can be expression vectors.

As used herein, the term “polypeptide” refers to a chain of amino acids of any length, regardless of modification (e.g., phosphorylation or glycosylation). In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, a “variant,” “mutant,” or “mutated” polynucleotide contains at least one polynucleotide sequence alteration as compared to the polynucleotide sequence of the corresponding wild-type or parent polynucleotide. Mutations may be natural, deliberate, or accidental. Mutations include substitutions, deletions, and insertions.

As used herein, “identity,” as known in the art, is a relationship between two or more polynucleotide or polypeptide sequences, as determined by comparing the sequences. In the art, “identity” also means the degree of sequence relatedness between the polynucleotide or polypeptide as determined by the match between strings of such sequences. “Identity” can also mean the degree of sequence relatedness of a polynucleotide or polypeptide compared to the full-length of a reference polynucleotide or polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

As used herein, “operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will assist the linked protein to be localized at the specific organelle.

As used herein, the terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

As used herein, “treat” means to prevent, reduce, decrease, or ameliorate one or more symptoms, characteristics or comorbidities of an age-related disease, disorder or condition; to reverse the progression of one or more symptoms, characteristics or comorbidities of an age related disorder; to halt the progression of one or more symptoms, characteristics or comorbidities of an age-related disorder; to prevent the occurrence of one or more symptoms, characteristics or comorbidities of an age-related disorder; to inhibit the rate of development of one or more symptoms, characteristics or comorbidities or combinations thereof.

As used herein, the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being affected.

As used herein, an “adjuvant” is a substance that increases the ability of an antigen to stimulate the immune system.

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

A used herein, the term “pharmaceutically-acceptable carrier” means one or more compatible solid or liquid fillers, diluents or encapsulating substances which are suitable for administration to a human or other vertebrate animal.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### II. Compositions

### A. Chimeric Fusion Proteins

Chimeric fusion proteins having immunodominant epitopes of RSV F and hMPV F are provided. Typically, the chimeric fusion protein includes an antigenic domain as referred to herein as chimeric polypeptide. Other optional domains of the fusion protein include, but are not limited to, a signal peptide, one or more cleavage sites, a trimerization domain, a purification tag, and one or more linkers. Residues for forming disulfide bridges (e.g., cysteines) are maintained or introduced. For example, in the exemplary antigenic proteins below, there are disulfides between the cleaved F1 and F2 fragments, and there are introduced disulfides to stabilize the protein in the pre-fusion conformation. In some embodiments, the chimeric fusion proteins are expressed and/or administered as a pre-fusion trimer that preserves structural features on key antigenic sites for both RSV and hMPV F proteins.

In some embodiments, the protein includes or consists of the antigenic domain or the antigenic domain and the multimerization/trimerization domain. In some embodiments, the signal peptide sequence, purification tag, and/or other domain(s) are cleaved, and the resulting mature protein is used for treating a subject in need thereof.

### 1. Antigenic Domain

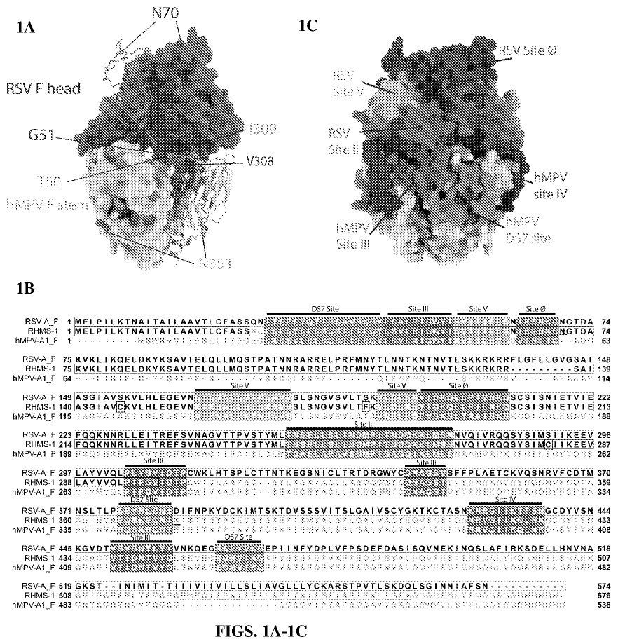

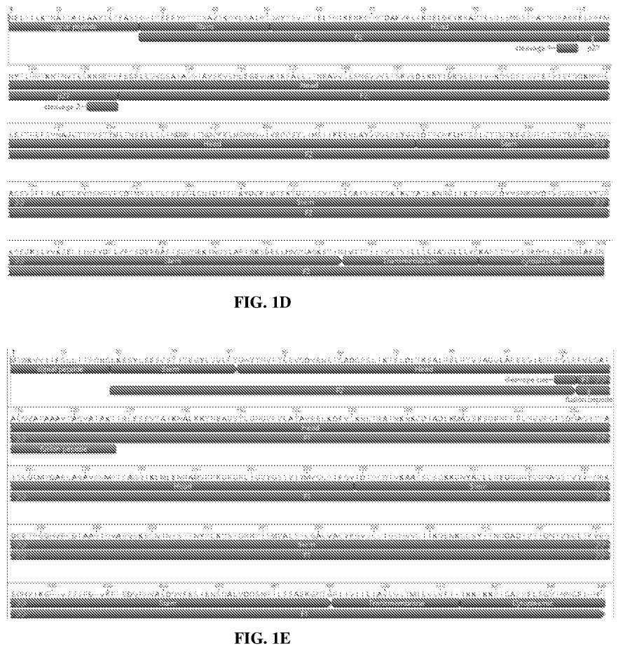

The antigenic domain typically includes one or more antigenic sites from RSV F, preferably the head of RSV F, and hMPV F, preferably the stem of hMPV F. In preferred embodiments, the chimeric fusion protein includes one or more, preferably all, of the RSV site Ø, RSV site V, RSV site II, hMPV site III, hMPV site IV, and hMPV DS7 sites. The sites can be identified in reference to the source sequences and/or an exemplary chimeric fusion protein expressly provided below. See also, FIGS. 1A-1C and SEQ ID NOS: 1, 3, 4 and 40.

a. Source Sequences

Reference sequences for RSV F (e.g., 5UDE and 5UDE)) and hMPV F (e.g., 5WB0) are known in the art and can be used as the origin sequences for the antigenic sites included in the antigenic domain. The source RSV F can be from RSV A or RSV B. See also FIG. 1B, which aligns RSV F and hMPV, and highlights the sequences of the antigenic sites.

An exemplary reference sequence for RSV F is MELPILKTNAITAILAAVTLCFASSQNITEEFYQTTCSAVSKGYLSALRTG WYTSVITIELSNIKENKCNGTDAKVKLIKQELDKYKSAVTELQLLMQSTPA TNNRARRELPRFMNYTLNNTKNTNVTLSKKRKRRFLGFLLGVGSAIASGIA VSKVLHLEGEVNKIKSALLSTNKAVVSLSNGVSVLTSKVLDLKNYIDKQLL PIVNKQSCSISNIETVIEFQQKNNRLLEITREFSVNAGVTTPVSTYMLTNS ELLSLINDMPITNDQKKLMSNNVQIVRQQSYSIMSIIKEEVLAYVVQLPLY GVIDTPCWKLHTSPLCTTNTKEGSNICLTRTDRGWYCDNAGSVSFFPLAET CKVQSNRVFCDTMNSLTLPSEVNLCNIDIFNPKYDCKIMTSKTDVSSSVIT SLGAIVSCYGKTKCTASNKNRGIIKTFSNGCDYVSNKGVDTVSVGNTLYYV NKQEGKSLYVKGEPIINFYDPLVFPSDEFDASISQVNEKINQSLAFIRKSD ELLHNVNAGKSTINIMITTIIIVIIVILLSLIAVGLLLYCKARSTPVTLSK DQLSGINNIAFSN (SEQ ID NO:3, RSV-A F). See also UniProtKB-Q6V2E7 (Q6V2E7_HRSV), Protein Data Base (PDB) No. 5UDE, UniProt P03420 (FUS_HRSVA), UniProt P11209 (FUS_HRSVR), PDB No. 5UDC, NCBI Reference Sequence: NC_001803 (Respiratory syncytial virus, complete genome) and NCBI Taxonomy ID: 11261 (Human respiratory syncytial virus strain RSS-2), each of which is specifically incorporated by reference herein in its entirety.

An exemplary reference sequence for hMPV F is
(SEQ ID NO: 4, hMPV-A1 F (furin-dFP))
MSWKVVIIFSLLITPQHGLKESYLEESCSTITEGYLSVLRIGWYINVFTLE
VGDVENLTCADGPSLIKTELDLTKSALRELRTVSADQLAREEQIENPRQSK
KRKRRVATAAAVTAGVAIAKTIRLESEVTAIKNALKKTNEAVSTLGNGVRV
LATAVRELKDFVSKNLTRAINKNKCDIADLKMAVSFSQFNRRFLNVVRQFS
DNAGITPAISLDLMIDAELARAVSNMPTSAGQIKLMLENRAMVRRKGFGIL
IGVYGSSVIYMVQLPIFGVIDTPCWIVKAAPSCSEKKGNYACLLREDQGWY
CQNAGSTVYYPNEKDCETRGDHVFCDTAAGINVAEQSKECNINISTTNYPC
KVSTGRHPISMVALSPLGALVACYKGVSCSIGSNRVGIIKQLNKGCSYITN
QDADTVTIDNTVYQLSKVEGEQHVIKGRPVSSSFDPVKFPEDQFNVALDQV
FESIENSQALVDQSNRILSSAEKGNTSGRENLYFQGGGGGSGYIPEAPRDQ
AYVRKDGEWVLLSTFLGGTEGRHHHHHH.

Another exemplary reference sequence for hMPV F is MSWKVVIIFSLLITPQHGLKESYLEESCSTITEGYLSVLRTGWYTNVFTLE VGDVENLTCADGPSLIKTELDLTKSALRELRTVSADQLAREEQIENPRQSR FVLGAIALGVATAAAVTAGVAIAKTIRLESEVTAIKNALKKTNEAVSTLGN GVRVLATAVRELKDFVSKNLTRAINKNKCDIADLKMAVSFSQFNRRFLNVV RQFSDNAGITPAISLDLMTDAELARAVSNMPTSAGQIKLMLENRAMVRRKG FGFLIGVYGSSVIYMVQLPIFGVIDTPCWIVKAAPSCSGKKGNYACLLRED QGWYCQNAGSTVYYPNEKDCETRGDHVFCDTAAGINVAEQSKECNINISTT NYPCKVSTGRHPISMVALSPLGALVACYKGVSCSIGSNRVGIIKQLNKGCS YITNQDADTVTIDNTVYQLSKVEGEQHVIKGRPVSSSFDPVKFPEDQFNVA LDQVFESIENSQALVDQSNRILSSAEKGNTGFIIVIILIAVLGSTMILVSV FIIIKKTKKPTGAPPELSGVTNNGFIPHN (SEQ ID NO:40, hMPV). See also UniProtKB-Q1A2Z0 (Q1A2Z0_9MONO) and NCBI Reference Sequence: NCBI Reference Sequence: NC_039199.1 (Human metapneumovirus isolate 00-1, complete genome), each of which is specifically incorporated by reference herein in its entirety.

b. Exemplary Antigenic Domains

In preferred embodiments, the antigenic domain include one or more, preferably all, of the following features: two cleavage sites of RSV; DsCav1 mutations (S155C, S190F, V207L, S290C relative to SEQ ID NO:3) (McLellan et al., Science (80)342:592-598 (2013), which is specifically incorporated by reference herein in its entirety); the fusion peptide of RSV F; replacement of part of the F2 N-terminus (residues 26-54 relative to SEQ ID NO:3) and the F1 C-terminus (residues 315-531 relative to SEQ ID NO:3) replaced by the homologous hMPV F sequences, with two junctions located on β2 and β7 strands; two glycosylation sites (RSV F-N70 relative to SEQ ID NO:3, hMPV F-N354 relative to SEQ ID NO:4 or hMPV F-N353 relative to SEQ ID NO:40). See also FIGS. 1A-1C, particularly 1B.

Exemplary antigenic domain sequences include HGLKESYLEESCSTITEGYLSVLRTGWYTSVITIELSNIKENKCNGTDAKV KLIKQELDKYKSAVTELQLLMQSTPATNNRARRELPRFMNYTLNNTKNTNV TLSKKRKRRSAIASGIAVCKVLHLEGEVNKIKSALLSTNKAVVSLSNGVSV LTFKVLDLKNYIDKQLLPILNKQSCSISNIETVIEFQQKNNRLLEITREFS VNAGVTTPVSTYMLTNSELLSLINDMPITNDQKKLMSNNVQIVRQQSYSIM CIIKEEVLAYVVQLPIFGVIDTPCWIVKAAPSCSEKKGNYACLLREDQGWY CQNAGSTVYYPNEKDCETRGDHVFCDTAAGINVAEQSKECNINISTTNYPC KVSTGRHPISMVALSPLGALVACYKGVSCSIGSNRVGIIKQLNKGCSYITN QDADTVTIDNTVYQLSKVEGEQHVIKGRPVSSSFDPVKFPEDQFNVALDQV FESIENSQALVDQSNRILSSAEKGNTSGRENLYFQ (SEQ ID NO:1), and fragments and variants thereof having, for example, at least 50, 60, 70, 75, 80, 85, 90, 95, or more percent sequence identity thereto.

In some embodiments, the sequences are modified to improve the stability and/or antigenicity of the antigenic domain or whole chimeric fusion protein. For example, interprotomer disulfides (IP-DSs) can be utilized to link protomers of the trimer. Results show that linking protomers of the hMPV F trimers in this way stabilized pre-fusion and post-fusion F proteins and elicited significantly higher neutralizing responses than the hMPV F proteins without IP-DSs (Stewart-Jones et al., Proc Natl Acad Sci 118 (2021), which is specifically incorporated by reference herein in its entirety).

### 2. Signal Peptide

The chimeric fusion proteins can include a signal peptide (also referred to as a signal sequence, targeting signal, localization signal, localization sequence, transit peptide, leader sequence or leader peptide), e.g., to assist in secretion of the protein during in vitro manufacture and/or in vivo expression. The signal peptide is cleaved off and is not typically part of the final active agent. Thus, chimeric fusion proteins with and without signal peptides, including the exemplary chimeric fusion protein sequence provided herein, are expressly provided with and without signal peptide sequences.

Signal peptides are short peptide sequences (usually 16-30 amino acids long), typically present at the N-terminal end of the protein. Thus, typically the signal peptide is N-terminal to the antigenic domain and can be at domain that is at the N-terminus of the chimeric fusion protein. The core of the signal peptide typically contains a stretch of hydrophobic amino acids (about 5-16 residues long) that has a tendency to form a single alpha-helix. In addition, many signal peptides begin with a positively charged stretch of amino acids, which may help to enforce proper topology of the polypeptide during translocation. At the end of the signal peptide there can be a stretch of amino acids that is recognized and cleaved by signal peptidase during or after completion of translocation to generate a free signal peptide and a mature protein.

The signal sequence of the chimeric fusion protein can be the signal sequence of e.g., RSV F, hMPV F, or an alternative heterologous signal sequence. In embodiments, the single sequence is one that facilitates secretion of chimeric fusion protein by the transfected or infected host cell.

Thus, in some embodiments, the RSV F or hMPV F signal peptide sequence is replaced by a signal peptide sequence that further improves its secretion from host cells.

An exemplary, non-limiting signal sequence is the signal peptide of RSV F (MELPILKTNAITAILAAVTLCFASS (SEQ ID NO:5)), which is utilized in the exemplary chimeric fusion protein constructs of the experiments described below. A non-limiting alternative signal peptide sequence is the signal peptide of hMPV F (MSWKVVIIFSLLITPQ (SEQ ID NO:6))

The fusion between the antigenic domain and the signal peptide sequence may further include or more additional amino acid residues. For example, bioinformatics analysis can be used to predict improved cleavage with different signal peptide sequences, and may include, for example, the incorporation of one, two, three, four, five or more additional amino acids added/inserted at the C-terminus of the signal peptide sequence before the start of the antigenic domain sequence. Likewise, in some embodiments, one, two, three, four, five or more additional amino acids are deleted/removed at the C-terminus of the signal peptide sequence before the start of the antigenic domain sequence.

### 3. Cleavage Sites

The chimeric protein can include one or more cleavage sites. For example, to become fusion competent, the F0 precursor of the naturally occurring viral F proteins typically must be cleaved into F1 and F2 subunits that are linked by two disulfide bonds to generate a mature meta-stable homotrimer. RSV F is cleaved at two furin cleavage sites with the p27 fragment in between F1 and F2 removed, whereas hMPV F has only one cleavage site that can be cleaved by the host membrane protease TMPRSS2. Thus, antigenic domain typically includes one, two, or more cleavage sites that facilitate maturation of the chimeric fusion protein.

Additionally, the chimeric fusion may additionally or alternatively include one or more additional cleavage domains inside or outside the antigenic domain. For example, in some embodiments, one or more additional cleavage domains are utilized to separate the antigenic domain and optionally but preferably the trimerization domain from other domains of the chimeric protein (e.g., the signal peptide, purification tag, etc.).

In the exemplary antigenic domain provided above and utilized as part of the chimeric fusion protein of the experiments below, furin cleavage sites are present at residues 81-84 (i.e., RARR (SEQ ID NO:7) and 106-111 (i.e., KKRKRR (SEQ ID NO:8)) relative to the amino acid sequence of SEQ ID NO:1. Such cleavage sites are also referred to as internal cleavage sites.

In the exemplary chimeric fusion protein utilized in the examples below, a TEV site (ENLYFQG (SEQ ID NO:9)) is present between the trimerization domain and the purification tag. Cleavage occurs between Q and G.

Other cleavage domains can be utilized e.g., by substitution including, for example, Furin cleavage sites have an RX(K/R)R consensus motif. As used herein, “X” or “x” in an amino acid sequence typically means any amino acid. See also, Zimmer, et al., J Virol., 76(18): 9218-9224 doi: 10.1128/JVI.76.18.9218-9224.2002 (2002), which is specifically incorporated by reference herein in its entirety. Thus, alternative Furin cleavage sites such as KRKRKKR (SEQ ID NO:11) can also be used.

Other metapneumovirus cleavage sites can also be used. For example, hMPV utilizes a sequence that includes RQSR (SEQ ID NO:12) by (see, e.g., van den Hoogen, et al., Virology, 295(1):119-132 (2002)).

Another exemplary protease cleavage site is a caspase-1 cleavage site, which may have a consensus motif of X-Glu-X-Asp (X-E-X-D). See, e.g., Shen, et al., Atherosclerosis, 210(2):422-429 (2010). doi:10.1016/j.atherosclerosis.2009.12.017.

The construct can additionally include, or one or more cleavage sites can be substituted with, one or more self-cleavage peptide sequences.

Exemplary self-cleaving peptides include, but are not limited to, 2A self-cleaving peptides. 2A self-cleaving peptides have a consensus motif DxExNPGP (SEQ ID NO:14), wherein “x” refers to any amino acid; and include, for example, Thosea asigna virus 2A peptide sequence including the sequence EGRGSLLTCGDVEENPGP (SEQ ID NO:15), P2A ATNFSLLKQAGDVEENPGP (SEQ ID NO:16), E2A QCTNYALLKLAGDVESNPGP (SEQ ID NO:17), and F2A VKQTLNFDLLKLAGDVESNPGP (SEQ ID NO:18). The cleavage is triggered by breaking of the peptide bond between the Proline (P) and Glycine (G) in C-terminal of 2A peptide, resulting in the peptide located upstream of the 2A peptide having extra amino acids on its C-terminal end while the peptide located downstream the 2A peptide has an extra Proline on its N-terminal end. Adding the optional linker, e.g., Gly-Ser-Gly on the N-terminal of a 2A peptide can help with efficiency.

Other exemplary cleavage sequence include, but are not limited to, RRRR (SEQ ID NO:38) and RRRRRR (SEQ ID NO:39).

### 4. Trimerization Domain

The chimeric fusion protein can include a multimerization domain, preferably a trimerization domain, that allows for trimerization (or other multimerization) of the chimeric fusion protein. The trimerization domain can be a heterologous or synthetic sequence.

The chimeric fusion protein utilized in the experiments below features a GCN4 trimerization domain having the amino acid sequence RMKQIEDKIEEILSKIYHIENEIARIKKLIGEA (SEQ ID NO:19). Many therapeutic proteins and protein subunit vaccines contain heterologous trimerization domains, and other multimerization domains are known in the art and can be used in addition or alternative to SEQ ID NO:19. See, e.g., Sliepen, et al., J Biol Chem., 290(12): 7436-7442 (2015), which is specifically incorporated by reference.

GCN4 variant sequences are also known and can include, e.g., amino acid sequences such as

(SEQ ID NO: 21)
RMKQIEDKIEEILSKIYHIENEIARIKKLIGERGGR;
(SEQ ID NO: 22)
RMKQIEDKIEEILSKIYHIENEIARIKKLIGNRTGGR;
(SEQ ID NO: 23)
RMKQIEDKIENITSKIYHIENEIARIKKLIGNRTGGR;
(SEQ ID NO: 24)
RMKQIEDKIEEILSKIYNITNEIARIKKLIGNRTGGR;
or
(SEQ ID NO: 25)
RMKQIEDKIENITSKIYNITNEIARIKKLIGNRTGGR;

any of which can optionally be preceded with NGT. The variant N residues relative to the convention GCN4 sequence can be used to link N-glycans that may reduce anti-GCN4 antibody response when utilizing the trimerization domain in vivo. See e.g., Sliepen, et al., J Biol Chem., 290(12): 7436-7442 (2015).

In addition to the GCN4-based isoleucine zipper (IZ), the T4 bacteriophage fibritin foldon (Fd) trimerization domain is also widely used in the art, and can likewise be substituted for GNC4. An amino acid sequence for this trimerization domain can be GSGYIPEAPRDGQAYVRKDGEWVLLSTFL (SEQ ID NO:26). See also Meier et al., J. Mol. Biol. 344, 1051-1069 (2004)).

Other suitable trimerization domains include the transmembrane and cytoplasmic tails of RSV F and hMPV F. These domains are illustrated in FIGS. 1D and 1E. An amino acid sequence for the RSV F transmembrane and cytoplasmic domains is IMITTIIIVIIVILLSLIAVGLLLYCKARSTPVTLSKDQLSGINNIAFSN (SEQ ID NO:44). An amino acid sequence for the hMPV F transmembrane and cytoplasmic domains is

(SEQ ID NO: 45)
FIIVIILIAVLGSTMILVSVFIIIKKTKKPTGAPPELSGVINNGFIPHN.

In some embodiments, the trimerization domain is or includes the amino acid sequence of any one of SEQ ID NOS:19-26 or 44-45, or a variant thereof with at least 50, 55, 60, 65, 70, 75, 80, 85, 90, 95, or more percent sequence identity thereto, preferably that that maintains the ability to induce chimeric fusion protein monomers to form trimers.

Other suitable trimerization domains include the trimerization domain of collagen and the HIV gp41 trimerization domain.

### 5. Peptide Linkers

The chimeric fusion protein can include one or more peptide linkers to e.g., separate various domains of the fusion protein.

Exemplary flexible linkers include, but are not limited to, Gly-Ser, Gly-Ser-Gly, Ala-Ser, Gly-Leu-Phe, Gly-Ser-Gly-Ser (SEQ ID NO:27), Gly-Gly-Gly-Ser (SEQ ID NO:28), Gly-Gly-Ser-Gly-Gly (SEQ ID NO:29), Gly-Gly-Gly-Gly-Ser (SEQ ID NO:30), (Gly₄-Ser)₂ (SEQ ID NO:31), (Gly₄-Ser)₄ (SEQ ID NO:32), (Gly-Gly-Gly-Gly-Ser)₃ (SEQ ID NO:33). The construct exemplified in the experiments below utilizes GLG linkers to link the antigenic domain and the trimerization domain as well as the trimerization domain and the TEV cleavage site, and a GSGG (SEQ ID NO:20) linker to link the TEV cleavage site to the 6× histidine purification tag.

### 6. Purification Tags and Reporters

The fusion protein can optionally include additional sequences or moieties, including, but not limited to purification tags, solubility enhancers, and/or reporters.

In some embodiments the purification tag is a polypeptide. Polypeptide purification tags are known in the art and include, but are not limited to His tags which typically include six or more, typically consecutive, histidine residues; green fluorescent protein (GFP), glutathione S-transferase (GST), polyhistidine, c-myc, hemagglutinin, Flag™ tag (Kodak, New Haven, CT), maltose E binding protein and protein A. More specific examples include FLAG tags including the sequence DYKDDDDK (SEQ ID NO:34); haemagglutinin (HA) tags including the sequence YPYDVP (SEQ ID NO:35); or MYC tags including the sequence ILKKATAYIL (SEQ ID NO:36) or EQKLISEEDL (SEQ ID NO:37). The fusion protein utilized in the experiments below includes 6 consecutive histidines at the C-terminus. Methods of using purification tags to facilitate protein purification are known in the art and include, for example, a chromatography step wherein the tag reversibly binds to a chromatography resin.

Although many proteins with therapeutic or commercial uses can be produced by recombinant organisms, the yield and quality of the expressed protein are variable due to many factors. For example, heterologous protein expression by genetically engineered organisms can be affected by the size and source of the protein to be expressed, the presence of an affinity tag linked to the protein to be expressed, codon biasing, the strain of the microorganism, the culture conditions of microorganism, and the in vivo degradation of the expressed protein. Some of these problems can be mitigated by fusing the protein of interest to an expression or solubility enhancing amino acid sequence. Exemplary expression or solubility enhancing amino acid sequences include maltose-binding protein (MBP), glutathione S-transferase (GST), thioredoxin (TRX), NUS A, ubiquitin (Ub), and a small ubiquitin-related modifier (SUMO).

In some embodiments, the compositions disclosed herein include expression or solubility enhancing amino acid sequence. In some embodiments, the expression or solubility enhancing amino acid sequence is cleaved prior administration of the composition to a subject in need thereof. The expression or solubility enhancing amino acid sequence can be cleaved in the recombinant expression system, or after the expressed protein in purified. In some embodiments, the expression or solubility enhancing is a ULP1 or SUMO sequence. Recombinant protein expression systems that incorporate the SUMO protein (“SUMO fusion systems”) have been shown to increase efficiency and reduce defective expression of recombinant proteins in E. coli., see for example Malakhov, et al., J. Struct. Funct. Genomics, 5: 75-86 (2004), U.S. Pat. Nos. 7,060,461, and 6,872,551. SUMO fusion systems enhance expression and solubility of certain proteins, including severe acute respiratory syndrome coronavirus (SARS-CoV) 3CL protease, nucleocapsid, and membrane proteins (Zuo et al., J. Struct. Funct. Genomics, 6:103-111 (2005)).

A reporter protein typically provides for some phenotypic change or enzymatic property. Examples of such proteins are provided in K. Weising et al. Ann. Rev. Genetics, 22, 421 (1988), and include, but are not limited to, carcinoembryonic antigen, secreted alkaline phosphatase, and the beta subunit of chorionic gonadotropin, glucuronidase (GUS), luciferase (e.g., Gaussia Luciferase (GLuc), Nanoluciferase (NLuc), and fluorescent proteins such as green fluorescent protein (GFP), enhanced green fluorescent protein (eGFP), turbo red fluorescent protein (TurboRFP), etc.

The reporter can serve as a measure or monitor of in vivo viral activity. For example, these reporters are released by cells infected with live virus into the blood, and can be measured peripherally to determine viral activity (Phuong, et al., Cancer Res., 63:2462-2469 (2003); Peng, et al., Nat. Med., 8:527-531 (2002); Shashkova, et al., Cancer Gene Ther., 15:61-72 (2008); Hiramatsu, et al., Cancer Science, 100, 1389-1396 (2005)).

Purifications tags, solubility enhancers, and reporters can be inserted anywhere in the fusions preferably where the do not disturb the ability of the fusion protein to induce an immune response against RSV and/or hMPV. Preferred locations include the N-terminus and/or C-terminus, but internal locations relative to other domains of the fusion protein are also contemplated.

### 7. Exemplary Chimeric Fusion Proteins

The experiments below utilize an exemplary chimeric fusion protein, also referred to as RSV head hMPV stem construct 1 (RHMS-1). RHMS-1 protein was stably expressed as a pre-fusion trimer that preserved the structural features on key antigenic sites for both RSV and hMPV F proteins. RHMS-1 retains immunodominant epitopes of both F proteins, including antigenic sites Ø, V, and II of RSV F, and sites IV, DS7, and III of hMPV F. Immunization of mice with RHMS-1 induced potent neutralizing antibodies that protected mice from both RSV and hMPV challenge.

The chimeric fusion proteins provided herein, such as RHMS-1, have several advantages over vaccination with pre-fusion RSV F or hMPV F, including a focus on recalling B cells to the most important protective epitopes and the ability to induce protection against two viruses with a single antigen. RHMS-1 was generated as a trimeric recombinant protein, and negative-stain EM analysis demonstrated the protein resembles the pre-fusion conformation. Probing of RHMS-1 antigenicity using a panel of RSV and hMPV F-specific monoclonal antibodies (mAbs) revealed the protein retains features of both viruses, including the pre-fusion site 0 epitope of RSV F. BALB/c mice immunized with RHMS-1 had serum binding and neutralizing antibodies to both viruses. RHMS-1 vaccinated mice challenged with RSV or hMPV had undetectable virus in lung homogenates for both viruses, in contrast to RSV F or hMPV F vaccinated mice, which had detectable virus for hMPV and RSV, respectively. Overall, these results show protection against two viruses with a single antigen.

A full, immature sequence for RHMS-1 is

(SEQ ID NO: 2)
MELPILKTNAITAILAAVTLCFASSHGLKESYLEESCSTITEGYLSVLRTG
WYTSVITIELSNIKENKCNGTDAKVKLIKQELDKYDSAVTELQLLMQSTPA

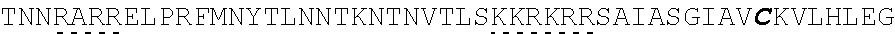

EVNKIKSALLSTNKAVVSLSNGVSVLTFKVLDLKNYIDKQLLPILNKQSCS
ISNIETVIEFQQKNNRLLEITREFSVNAGVTTPVSTYMLTNSELLSLINDM
PITNDQKKLMSNNVQIVRQQSYSIMCIIKEEVLAYVVQLPIFGVIDTPCWI
VKAAPSCSEKKGNYACLLREDQGWYCQNAGSTVYYPNEKDCETRGDHVFCD
TAAGINVAEQSKECNINISTTNYPCKVSTGRHPISMVALSPLGALVACYKG
VSCSIGSNRVGIIKQLNKGCSYITNQDADTVTIDNTVYQLSKVEGEQHVIK
GRPVSSSFDPVKFPEDQFNVALDQVFESIENSQALVDQSNRILSSAEKGNT
SGRENLYFQGSGRMKQIEDKIEEILSKIYHIENEIARIKKLIGEAGSGREN
LYFQGGSGGHHHHHH.

RHMS-1 features an N-terminal signal peptide sequence (amino acids 1-25); an antigenic domain composed of two segments derived from hMPV-A1 F (amino acids 26-51 and 294-519, annotated with double underlining) fused to two intervening segments derived from RSV-A F (amino acids 26-136 and 137-293 including four Ds-Cav1 mutations in bolded italics; and two cleavage sites in dotted underlining); a SG linker (amino acids 521-522); a GCN4 trimerization domain (amino acids 523-555 in bold); a GSG linker (amino acids 556-558); a TEV cleavage site (amino acids 560-566 in italics); a GSGG (SEQ ID NO:20) linker (amino acids 567-570), and a six histidine purification tag (amino acids 571-576). A second TEV cleavage site can be found at the end of the segment derived from hMPB-A1 F and before the GCN4 trimerization domain (amino acids 514-520 in italics).

In some embodiments, part or all of the peptide signal sequence and/or the purification tag and/or trimerization domain are absent. In some embodiments, the chimeric fusion protein includes the sequence

(SEQ ID NO: 41)
HGLKESYLEESCSTITEGYLSVLRTGWYTSVITIELSNIKENKCNGTDAKV

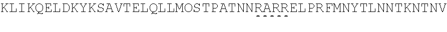

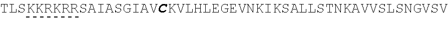

LTFKVLDLKNYIDKQLLPILNKQSCSISNIETVIEFQQKNNRLEEITREFS
VNAGVTTPVSTYMLTNSELLSLINDMPITNDQKKLMSNNVQIVRQQSYSIM
CIIKEEVLAYVVQLPIFGVIDTPCWIVKAAPSCSEKKGNYACLLREDQGWY
CQNAGSTVYYPNEKDCETRGDHVFCDTAAGINVAEQSKECNINISTTNYPC
KVSTGRHPISMVALSPLGALVACYKGVSCSIGSNRVGIIKQLNKGCSYITN
QDADTVTIDNTVYQLSKVEGEQHVIKGRPVSSSFDPVKFPEDQFNVALDQV
FESIENSQALVDQSNRILSSAEKGNTSGRENLYFQGSGRMKQIEDKIEEIL
SKIYHIENEIARIKKLIGEA, or
(SEQ ID NO: 42)
MELPILKTNAITAILAAVTLCFASSHGLKESYLEESCSTITEGYLSVLRTG
WYTSVITIELSNIKENKCNGTDAKVKLIKQELDKYKSAVTELQLLMQSTPA

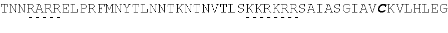

EVNKIKSALLSTNKAVVSLSNGVSVLTFKVLDLKNYIDKQLLPILNKQSCS
ISNIETVIEFQQKNNRLLEITREFSVNAGVTTPVSTYMLTNSELLSLINDM
PITNDQKKLMSNNVQIVRQQSYSIMCIIKEEVLAYVVQLPIFGVIDTPCWI
VKAAPSCSEKKGNYACLLREDQGWYCQNAGSTVYYPNEKDCETRGDHVFCD
TAAGINVAEQSKECNINISTTNYPCKVSTGRHPISMVALSPLGALVACYKG
VSCSIGSNRVGIIKQLNKGCSYITNQDADTVTIDNTVYQLSKVEGEQHVIK
GRPVSSSFDPVKFPEDQFNVALDQVFESIENSQALVDQSNRILSSAEKGNT
SGRENLYFQGSGRMKQIEDKIEEILSKIYHIENEIARIKKLIGEA, or
(SEQ ID NO. 43)
HGLKESYLEESCSTITEGYLSVLRTGWYTSVITIELSNIIKENKCNGTDAKV

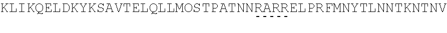

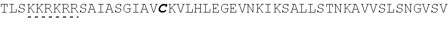

LTFKVLDLKNYIDKQLLPILNKQSCSISNIETVIEFQQKNNRLLEITREFS
VNAGVTTPVSTYMLTNSELLSLINDMPITNDQKKLMSNNVQIVRQQSYSIM
CIIKEEVLAYVVQLPIFGVIDTPCWIVKAAPSCSEKKGNYACLLREDQGWY
CQNAGSTVYYPNEKDCETRGDHVFCDTAAGINVAEQSKECNINISTTNYPC
KVSTGRHPISMVALSPLGALVACYKGVSCSIGSNRVGIIKQLNKGCSYITN
QDADTVTIDNTVYQLSKVEGEQHVIKGRPVSSSFDPVKFPEDQFNVALDQV
FESIENSQALVDQSNRILSSAEKGNTSGRENLYFQGSGRMKQIEDKIEEIL
SKIYHIENEIARIKKLIGEAGSGRENLYFQGGSGGHHHHHH, or
(SEQ ID NO: 46)
HGLKESYLEESCSTITEGYLSVLRTGWYTSVITIELSNIKENKCNGTDAKV

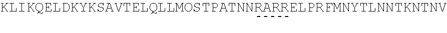

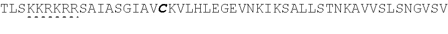

LTFKVLDLKNYIDKQLLPILNKQSCSISNIETVIEFQQKNNRLLEITREFS
VNAGVTTPVSTYMLTNSELLSLINDMPITNDQKKLMSNNVQIVRQQSYSIM
CIIKEEVLAYVVQLPIFGVIDTPCWIVKAAPSCSEKKGNYACLLREDQGWY
CQNAGSTVYYPNEKDCETRGDHVFCDTAAGINVAEQSKECNINISTTNYPC
KVSTGRHPISMVALSPLGALVACYKGVSCSIGSNRVGIIKQLNKGCSYITN
QDADTVTIDNTVYQLSKVEGEQHVIKGRPVSSSFDPVKFPEDQFNVALDQV
FESIENSQALVDQSNRILSSAEKGNTSGRENLYFQGSGRMKQIEDKIEEIL
SKIYHIENEIARIKKLIGEAGSGRENLYFQ, or
(SEQ ID NO: 47)
MELPILKTNAITAILAAVTLCFASSHGLKESYLEESCSTITEGYLSVLRTG
WYTSVITIELSNIKENKCNGTDAKVKLIKQELDKYKSAVTELQLLMQSTPA

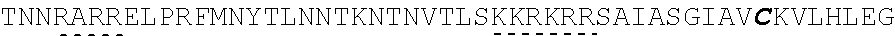

EVNKIKSALLSTNKAVVSLSNGVSVLTFKVLDLKNYIDKQLLPILNKQSCS
ISNIETVIEFQQKNNRLLEITREFSVNAGVTTPVSTYMLTNSELLSLINDM
PITNDQKKLMSNNVQIVRQQSYSIMCIIKEEVLAYVVQLPIFGVIDTPCWI
VKAAPSCSEKKGNYACLLREDQGWYCQNAGSTVYYPNEKDCETRGDHVFCD
TAAGINVAEQSKECNINISTTNYPCKVSTGRHPISMVALSPLGALVACYKG
VSCSIGSNRVGIIKQLNKGCSYITNQDADTVTIDNTVYQLSKVEGEQHVIK
GRPVSSSFDPVKFPEDQFNVALDQVFESIENSQALVDQSNRILSSAEKGNT
SGRENLYFQGSGRMKQIEDKIEEILSKIYHIENEIARIKKLIGEAGSGREN
LYFQ.

Variants and fragments are also provided. Thus, in some embodiments, the chimeric fusion protein is or includes SEQ ID NO:2 or any one of SEQ ID NOS:41-43 or 46-47, or a fragment or variant thereof with at least 50, 55, 60, 65, 70, 75, 80, 85, 90, or 95 percent sequence identity thereto. In some embodiments, one or more of the domains is absent, substituted, or otherwise modified as discussed above.

There are disulfides between the cleaved F1 and F2 fragments, and there are introduced disulfides to stabilize the protein in the pre-fusion conformation. The fusion protein can be used in monomeric to multimeric (e.g., trimeric) form.

### B. Isolated Nucleic Acids

All of the accession numbers provided herein are specifically incorporated by reference herein in their entireties. Where an amino acid (e.g., polypeptide) is expressly provided or provided in an accession number, all nucleic acid sequences including but not limited to, gene, cDNA, and mRNA sequences and their complements, and codon variations thereof encoding the amino acid sequence, both as single strands and double strands, and in any form of nucleic acid, including, but not limited to DNA and RNA, and analogs and variations thereof including but limited to modified bases, sugars, and linkages (e.g., peptide nucleic acids (PNA)), are all expressly provided.

Isolated nucleic acid sequences encoding the chimeric fusion proteins and individual domains and fragments thereof, and vectors and other expression constructs encoding the foregoing are also disclosed herein. As used herein, “isolated nucleic acid” refers to a nucleic acid that is separated from other nucleic acid molecules that are present in a mammalian genome, including nucleic acids that normally flank one or both sides of the nucleic acid in a mammalian genome. The term “isolated” as used herein with respect to nucleic acids also includes the combination with any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

The nucleic acid sequences encoding the disclosed proteins and polypeptides can be or include, for example, engineered genomic sequences and fragments of naturally occurring genomic sequence, mRNA sequence wherein the exons have been deleted, and other nucleic acid sequences. Nucleic acids encoding the chimeric fusion proteins and domains thereof may be optimized for expression in the expression host of choice. Codons may be substituted with alternative codons encoding the same amino acid to account for differences in codon usage different host organisms. In this manner, the nucleic acids may be synthesized using expression host-preferred codons.

Nucleic acids can be in sense or antisense orientation, or can be complementary to a reference sequence encoding the chimeric fusion protein or domain(s) thereof. Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Common modifications are discussed in more detail below.

Nucleic acids encoding polypeptides can be administered to subjects in need thereof. Nucleic delivery involves introduction of “foreign” nucleic acids into a cell and ultimately, into a live animal. Compositions and methods for delivering nucleic acids to a subject are known in the art (see Understanding Gene Therapy, Lemoine, N. R., ed., BIOS Scientific Publishers, Oxford, 2008).

### 1. Vectors and Host Cells

Vectors encoding chimeric fusion proteins and domains thereof are also provided. Nucleic acids, such as those described above, can be inserted into vectors for expression in cells. As used herein, a “vector” is a replicon, such as a plasmid, phage, virus or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. For example, the control sequence can be incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA).

An expression vector can include a tag sequence. Tag sequences are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, CT), maltose E binding protein and protein A.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as insect cells or mammalian cells (e.g., CHO cells) can be used to, for example, produce the fusion proteins described herein.

The vectors can be used to express fusion protein nucleic acids in cells. An exemplary vector includes, but is not limited to, an adenoviral vector. One approach includes nucleic acid transfer into primary cells in culture followed by autologous transplantation of the ex vivo transformed cells into the host, either systemically or into a particular organ or tissue. Ex vivo methods can include, for example, the steps of harvesting cells from a subject, culturing the cells, transducing them with an expression vector, and maintaining the cells under conditions suitable for expression of the encoded polypeptides. These methods are known in the art of molecular biology. The transduction step can be accomplished by any standard means used for ex vivo gene therapy, including, for example, calcium phosphate, lipofection, electroporation, viral infection, and biolistic gene transfer. Alternatively, liposomes or polymeric microparticles can be used. Cells that have been successfully transduced then can be selected, for example, for expression of the coding sequence or of a drug resistance gene. The cells then can be lethally irradiated (if desired) and injected or implanted into the subject. In one embodiment, expression vectors containing nucleic acids encoding fusion proteins are transfected into cells that are administered to a subject in need thereof.

In vivo nucleic acid therapy can be accomplished by direct transfer of a functionally active RNA or DNA into mammalian somatic tissue or organ in vivo. Nucleic acids may also be administered in vivo by viral means. Nucleic acid molecules encoding polypeptides or fusion proteins may be packaged into retrovirus vectors using packaging cell lines that produce replication-defective retroviruses, as is well-known in the art. Other virus vectors may also be used, including recombinant adenoviruses and vaccinia virus, which can be rendered non-replicating. In addition to naked DNA or RNA (e.g., mRNA), or viral vectors, or engineered bacteria may be used as vectors.

Nucleic acids may also be delivered by other carriers, including liposomes, polymeric micro- and nanoparticles, including but not limited to polymeric, liposomal, and protein nanoparticles, and polycations.

In addition to virus- and carrier-mediated gene transfer in vivo, physical means well-known in the art can be used for direct transfer of DNA, including administration of plasmid DNA and particle-bombardment mediated gene transfer.

### 2. Oligonucleotide Composition

The disclosed nucleic acids nucleic acids can be DNA or RNA nucleotides which typically include a heterocyclic base (nucleic acid base), a sugar moiety attached to the heterocyclic base, and a phosphate moiety which esterifies a hydroxyl function of the sugar moiety. The principal naturally-occurring nucleotides include uracil, thymine, cytosine, adenine and guanine as the heterocyclic bases, and ribose or deoxyribose sugar linked by phosphodiester bonds.

In some embodiments, the oligonucleotides are composed of nucleotide analogs that have been chemically modified to improve stability, half-life, or specificity or affinity for a target receptor, relative to a DNA or RNA counterpart. The chemical modifications include chemical modification of nucleobases, sugar moieties, nucleotide linkages, or combinations thereof. As used herein ‘modified nucleotide” or “chemically modified nucleotide” defines a nucleotide that has a chemical modification of one or more of the heterocyclic base, sugar moiety or phosphate moiety constituents. In some embodiments, the charge of the modified nucleotide is reduced compared to DNA or RNA oligonucleotides of the same nucleobase sequence. For example, the oligonucleotide can have low negative charge, no charge, or positive charge.

Typically, nucleoside analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). In some embodiments, the analogs have a substantially uncharged, phosphorus containing backbone.

### 3. Delivery Vehicles

The disclose compounds can be administered and taken up into the cells of a subject with or without the aid of a delivery vehicle. Appropriate delivery vehicles for the disclosed compositions are known in the art and can be selected to suit the particular composition. For example, if the compound is a nucleic acid or vector, the delivery vehicle can be a viral vector, for example a commercially available preparation, such as an adenovirus vector (Quantum Biotechnologies, Inc. (Laval, Quebec, Canada). The viral vector delivery can be via a viral system, such as a retroviral vector system which can package a recombinant retroviral genome (see e.g., Pastan et al., (1988) Proc. Natl. Acad. Sci. U.S.A. 85:4486; Miller et al., (1986) Mol. Cell. Biol. 6:2895). The recombinant retrovirus can then be used to infect and thereby deliver to the infected cells nucleic acid encoding the compound. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948 (1994)), adeno-associated viral (AAV) vectors (Goodman et al., Blood 84:1492-1500 (1994)), lentiviral vectors (Naidini et al., Science 272:263-267 (1996)), pseudotyped retroviral vectors (Agrawal et al., Exper. Hematol. 24:738-747 (1996)).

Physical transduction techniques can also be used, such as liposome delivery and receptor-mediated and other endocytosis mechanisms (see, for example, Schwartzenberger et al., Blood 87:472-478 (1996)). For example in some embodiments, the composition is delivered via a liposome. Commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art are well known. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.). This disclosed compositions and methods can be used in conjunction with any of these or other commonly used gene transfer methods.

In some embodiments, the nucleic acid or protein is incorporated into or encapsulated by a nanoparticle or microparticle (e.g., polymeric, liposomal, protein, etc.), micelle, synthetic lipoprotein particle, or carbon nanotube. For example, the compositions can be incorporated into a vehicle such as polymeric or protein microparticles or nanoparticles which provide protection and/or controlled release of the compound. In some embodiments, release of the drug(s) is controlled by diffusion of the compound out of the microparticles or nanoparticles and/or degradation of the particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof.

### C. Recombinant Viruses

RSV and hMPV are non-segmented negative-sense single-stranded RNA viruses. The genome of RSV is 15.2 kb long, having ten genes encoding 11 proteins: 3′-NS1-NS2-N-P-M-SH-G-F-M2-L-5′. The genomic organization of HMPV is similar to RSV; however, hMPV lacks the non-structural genes, NS1 and NS2, and the hMPV antisense RNA genome contains eight open reading frames in slightly different gene order than RSV: 3′-N-P-M-F-M2-SH-G-L-5′.

Methods of engineering recombinant negative-sense single-stranded RNA viruses, including for RSV and hMPV are known in the art. See, e.g., Biacchesi, et al., Virology, 315:1-9 (2003), Biacchesi, et al., J. Virology, 78(23):12877-12887 (2004), Collins, et al., Proc. Natd. Acad. Sci. U.S.A 92:11563-11567 (1995), Luongo, et al., 86(19): 10792-10804 (2012) each of which is specifically incorporated by reference herein in its entirety. Briefly, recombinant DNA can be transcribed by T7 RNA polymerase to generate a full-length positive-strand RNA complimentary to the viral genome. Expression of this RNA in cells also expressing helper proteins results in production of reconstituted recombinant virus. For example, recombinant RSV may be produced by expressing a first expression vector including a polynucleotide encoding a recombinant RSV antigenome and a second expression vector including a polynucleotide encoding one or more protein selected from a group consisting of N, P, L and M2-1 proteins together. In this way, RSV and hMPV can be engineered to express variant proteins such as the chimeric fusion proteins disclosed herein. Thus, recombinant RSV and hMPV constructs, viral vectors, genomes, antigenomes, and live or inactivated recombinant viruses encoding a chimeric fusion protein, optionally but preferably in place of the F protein, are all provided. In some embodiments, the chimeric fusion protein is cloned into an attenuated RSV or hMPV background. Examples of attenuated RSV and hMPV that serve as a background for introduction and/or substitution of a chimeric fusion protein are known in the art. See, for example, U.S. Published Application Nos. 2021/0188920, 20210330782, and 2019/0192592 each of which are specifically incorporated by reference herein in their entireties. The recombinant viruses can be inactivated, but are preferably live viruses. In some embodiments, the viruses are replication competent. Such recombinant virus typically have a genome that encodes the chimeric fusion protein and can produce virus carrying the chimeric fusion protein in infected host cells. The virus may also be replication incompetent. Such viruses can carry the chimeric fusion, but are not capable of producing new virus in infected cells.

### D. Formulations

The disclosed compounds can be formulated in a pharmaceutical composition. Pharmaceutical compositions can be for administration by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration.

The compositions can be administered locally or systemically.

### 1. Formulations for Parenteral Administration

Compounds and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as POLYSORBATE® 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

Recombinantly modified virus can also be introduced into a host with a physiologically acceptable carrier and/or adjuvant. Useful carriers are well known in the art, and include, e.g., water, buffered water, 0.4% saline, 0.3% glycine, hyaluronic acid and the like.

### 2. Formulations for Mucosal and Pulmonary Administration

Active agent(s) and compositions thereof can be formulated for pulmonary or mucosal administration. The administration can include delivery of the composition to the lungs, nasal, oral (sublingual, buccal), vaginal, or rectal mucosa. In a particular embodiment, the composition is formulated for and delivered to the subject sublingually.

In one embodiment, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions composed of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, CA).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different active agents may be administered to target different regions of the lung in one

### E. Immunogenic Compositions and Vaccines

Immunogenic compositions and vaccines are also provided. Typically, an immunogenic composition includes an adjuvant, an antigen (which may be e.g., a chimeric fusion protein, a nucleic acid encoding the same, or a virus having or encoding the fusion protein), or a combination thereof. The combination of an adjuvant and an antigen can be referred to as a vaccine. When administered to a subject in combination, the adjuvant and antigen can be administered in separate pharmaceutical compositions, or they can be administered together in the same pharmaceutical composition. The disclosed chimeric fusion proteins and nucleic acids encoding the same (e.g., mRNA) can serve as the antigen component of an immunogenic composition or vaccine formulation. In some embodiments of the antigen provided as a recombinant protein, the chimeric fusion protein is a monomeric form. In preferred embodiments of antigen provided as a recombinant protein, the chimeric fusion protein is a multimeric, preferably trimeric, form. Additionally, the composition can include an adjuvant. Thus, in some embodiments, the composition includes both an antigen and an adjuvant. Two or more different antigens, one or more different adjuvants, or combinations thereof, can be used or combined. In particular embodiments, the formulation is a an adjuvant+protein or a nanoparticle-based vaccines with or without adjuvant and using mRNA or DNA as the means of delivering antigen.

### 1. Antigens and Virus

Antigens are compounds that are specifically bound by antibodies or T lymphocyte antigen receptors. They stimulate production of or are recognized by antibodies. Sometimes antigens are part of the host itself in an autoimmune disease. An immunogen is an antigen (or adduct) that is able to trigger a humoral or cell-mediated immune response. It first initiates an innate immune response, which then causes the activation of the adaptive immune response. An antigen binds the highly variable immunoreceptor products (B cell receptor or T cell receptor) once these have been generated. Immunogens are those antigens, termed immunogenic, capable of inducing an immune response. Thus, an immunogen is necessarily an antigen, but an antigen may not necessarily be an immunogen. For brevity, the disclosed antigenic and vaccines composition are typically referred to as having or encoding an antigen. However, unless specifically indicated otherwise, any of the antigens can also be an immunogenic (i.e., an immunogen). Thus, all the disclosure of compositions and methods of use related to antigenic compositions and vaccines is also expressly provided with respect to immunogen unless indicated to the contrary.

In some embodiments, the antigenic or vaccine composition includes an effective amount of a live (e.g., attenuated) or inactivated virus that has and/or encodes a chimeric fusion protein that serves as an antigen when administered to the subject.

Thus, immunogenic compositions and vaccine including an effective amount a chimeric fusion protein, or a nucleic acid or virus having or encoding the same, to induce an immune response thereto are provided. The fusion protein can also be post-translationally modified including one or more of cleavage to remove a signal sequence and/or a purification domain and/or one or more other domains, to cleave the chimeric protein at other cleavage sites (e.g., one or more internal cleavage sites such those discussed above), and/or to form a multimer (e.g., a trimer). The immunogenic compositions and vaccines can be used in methods of treating and preventing viral infections.

### 2. Adjuvants

Immunologic adjuvants stimulate the immune system's response to a target antigen, but do not provide immunity themselves. Adjuvants can act in various ways in presenting an antigen to the immune system. Adjuvants can act as a depot for the antigen, presenting the antigen over a longer period of time, thus maximizing the immune response before the body clears the antigen. Examples of depot type adjuvants are oil emulsions. An adjuvant can also act as an irritant, which engages and amplifies the body's immune response.

The adjuvant may be without limitation AS03, AddaSO3, MF59, CpG, alum (e.g., aluminum hydroxide, aluminum phosphate); saponins purified from the bark of the Q. saponaria tree such as Quil A (a mixture of more than 25 different saponin molecules), or subcombinations or individual molecules thereof such as QS21 (a glycolipid that elutes in the 21st peak with HPLC fractionation; Antigenics, Inc., Worcester, Mass.); poly[di(carboxylatophenoxy) phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic).

Adjuvants may be TLR ligands. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

The adjuvant can also be oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminum salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-.gamma.), macrophage colony stimulating factor, and tumor necrosis factor.

Immunostimulatory complexes called ISCOMs are particulate antigen delivery systems having antigen, cholesterol, phospholipid and saponin (Quil A or other saponin) with potent immunostimulatory activity. ISCOMATRIX® is a particulate adjuvant having cholesterol, phospholipids and saponins (Quil A) but without containing antigen. See, e.g., U.S. Pat. No. 9,149,520, Sun, et al., Volume 27, Issue 33, 16 Jul. 2009, Pages 4388-4401, and Morelli, et al., J Med Microbiol. 2012 July; 61(Pt 7):935-43. doi: 10.1099/jmm.0.040857-0. Epub 2012 Mar. 22. This adjuvant has principally the same structure as ISCOMs, consisting of perforated cage-like particles of approximately 40 nm in diameter. The antigens can be formulated with ISCOMATRIX® to produce vaccines capable of antigen presentation and immunostimulants similar to ISCOMs-type formulations, but with a wider range of applicability, since its use is not limited to hydrophobic membrane proteins. Modifications of ISCOMs formulations and ISCOMATRIX® have also been developed to achieve a better association of some antigens, such as described in WO 98/36772.

ISCOMs and ISCOMATRIX® combine the advantages of a particulate delivery system with the in situ presence of an adjuvant (Quil A) and consequently have been found to be more immunogenic than other colloidal systems such as liposomes and protein micelles. Formulations of ISCOMs and ISCOMATRIX® retained the adjuvant activity of the Quil A, while increasing its stability, reducing its hemolytic activity, and producing less toxicity. They also generate a similar immune response to the one obtained by immunizing with simple mixtures of antigen and saponin, but allow for the use of substantially smaller amounts of antigen. Several ISCOMs-type vaccine formulations or containing ISCOMATRIX® have been approved for veterinary use, for example against equine influenza virus.

Other liposomal systems mainly composed of saponins from Q. saponaria and sterols (primarily cholesterol) have been described, one of which is referred to as ASO1B. See, e.g., WO 96/33739, being also formulated as emulsions such as described in US 2005/0220814. See, also, U.S. Published Application No. 2011/0206758.

Iscomatrix-like adjuvants such as ISCOMATRIX® are thought to function via canonical inflammasome activation and subsequent release of pro-inflammatory cytokines such as IL-18 and IL-10 (Wilson, et al., Journal of immunology. 2014; 192(7):3259-68. doi: 10.4049/jimmunol.1302011. PubMed PMID: 24610009). This mechanism is thought to be mediated at least in-part by endosomal degradation and the release of NRLP3-activating cathepsin proteases into the cytosol.

### III. Methods of Use

### A. Methods of Treatment

Methods of inducing an immune response in a subject (e.g., a human) by administering to the subject a therapeutically effective amount of a disclosed immunogenic or vaccine composition are provided. The immune response can be induced, increased, or enhanced by the composition compared to a control (e.g., absence of the composition or presence of another composition). The composition can include an effective amount of a chimeric fusion protein as monomer or multimer such as trimer, a nucleic acid encoding the chimeric fusion protein such as a viral vector or mRNA, a recombinant virus having and/or encoding the chimeric fusion protein, or any combination hereof. Adjuvant can optionally be delivered together or separately. In some embodiment the components, particular nucleic acids encoding the chimeric fusion protein (e.g., mRNA or DNA) are delivered using nanoparticles, e.g., polymeric or liposomal nanoparticles. The immune response is typically against the chimeric fusion protein, and thus preferably increases immunity against one or more, preferably two or more pneumoviruses including, but not limited to RSV and hMPV.

In some embodiments, the disclosed compositions increase a B cell response. In some embodiments, a disclosed composition is administered to a subject in need thereof in an effective amount to induce an antigen-specific antibody response (e.g., IgG, IgG2a, IgG1, or a combination thereof), increase a response in germinal centers, increase plasmablast frequency, increase inflammatory cytokine expression, or a combination thereof.

In some embodiments, the administration of the composition alternatively or additionally induces a B-memory cell response in subjects administered the composition compared to a control. A B-memory cell response is intended to mean an increased frequency of peripheral blood B lymphocytes capable of differentiation into antibody-secreting plasma cells upon antigen encounter.

In some embodiments, the compositions can induce an effector cell response such as a CD4 or CD8 T-cell immune response, against at least one of the component antigen(s) or antigenic compositions compared to the effector cell response obtained under control conditions (e.g., absence of the composition or presence of another composition). The term “improved effector cell response” refers to a higher effector cell response such as a CD8 or CD4 response obtained in a subject after administration of a disclosed composition than that obtained under control conditions.

The described compositions may be administered as part of prophylactic vaccines or immunogenic compositions which confer resistance in a subject to subsequent exposure to infectious agents, or as part of therapeutic vaccines, which can be used to initiate or enhance a subject's immune response to a pre-existing antigen.

The desired outcome of a prophylactic or therapeutic immune response may vary according to the disease or condition to be treated, or according to principles well known in the art. For example, an immune response against an infectious agent may completely prevent colonization and replication of an infectious agent, affecting “sterile immunity” and the absence of any disease symptoms. However, a vaccine against infectious agents may also be considered effective if it reduces the number, severity or duration of symptoms; if it reduces the number of individuals in a population with symptoms; or reduces the transmission of an infectious agent. Similarly, immune responses may completely treat a disease, may alleviate symptoms, or may be one facet in an overall therapeutic intervention against a disease.

The disclosed compositions may be used in methods of inducing protective immunity against an infectious agent, disease, or condition by administering to a subject (e.g., a human) a therapeutically effective amount of the compositions. “Protective immunity” or “protective immune response” refers to immunity or eliciting an immune response against an infectious agent, which is exhibited by a subject (e.g., a human), that prevents or ameliorates an infection or reduces at least one symptom thereof.

In some embodiments, the methods include inducing the production of neutralizing antibodies or inhibitory antibodies in a subject (e.g., a human) by administering any of the disclosed compositions to the subject. In some embodiments, a disclosed composition is administered to a subject in need thereof in an effective amount to increase an antigen-specific antibody response (e.g., IgA, IgD, IgE, IgM, IgG, IgG2a, IgG1, or a combination thereof). The antibody response is important for preventing many infections and may also contribute to resolution of infection. For example, when a vertebrate (e.g., a human) is infected with a virus, antibodies are produced against many epitopes on multiple virus proteins. A subset of these antibodies can block virus infection by a process called neutralization. Antibodies can neutralize viral infectivity in a number of ways. They may interfere with virion binding to receptors (blocking viral attachment), block uptake into cells (e.g., blocking endocytosis), prevent uncoating of the genomes in endosomes, or cause aggregation of virus particles. Many enveloped viruses are lysed when antiviral antibodies and serum complement disrupt membranes.

Upon administration of an immunogenic or vaccine composition as described herein, e.g., via injection, aerosol, droplet, oral, topical or other route, the immune system of the host responds to the composition by producing antibodies specific for the chimeric fusion protein. As a result of the vaccination the host becomes at least partially or completely immune to pneumoviruses such as RSV and/or hMPV, preferably both RSV and hMPV infection, or resistant to developing mild, moderate, or severe disease caused by pneumoviruses such RSV and/or hMPV, particularly of the lower respiratory tract.

The host to which the compositions can be administered can be any mammal susceptible to infection by a pneumovirus such as RSV and/or hMPV or a closely related virus, and capable of generating a protective immune response to antigens of the virus. Thus, suitable hosts include humans, non-human primates, bovine, equine, swine, ovine, caprine, lagamorph, rodents, such as mice or cotton rats, etc. Accordingly, the disclosure provides methods for creating vaccines for a variety of human and veterinary uses.

The compositions can be administered to a subject susceptible to or otherwise at risk of pneumoviruses infection in an “immunogenically effective dose” which is sufficient to induce or enhance the individual's immune response capabilities against one, or preferably both, viruses. The compositions can be administered to a subject via injection, aerosol delivery, nasal spray, nasal droplets, oral inoculation, or topical application.

In particular embodiments, the composition includes an immunogenically effective amount of a chimeric fusion protein, or a nucleic acid (e.g., mRNA or viral vector) encoding the same.

In other embodiments, the composition includes an immunologically effective amount of a recombinant virus having and/or encoding the chimeric fusion protein. The virus can be live or inactivated, can be replication competent or incompetent (i.e., non-replicating), and can be administered in any suitable means consistent with the nature of the virus. For example, live virus can be administered according to well established human RSV vaccine protocols (Karron, et al., JID 191:1093-104, (2005)). In some embodiments, adults or children can be inoculated intranasally via droplet with an immunogenically effective dose of virus, typically in a volume of 0.5 ml of a physiologically acceptable diluent or carrier. This has the advantage of simplicity and safety compared to an alternative parenteral immunization approach, e.g., with a non-replicating vaccine. It also provides direct stimulation of local respiratory tract immunity, which plays a major role in resistance to RSV. Further, this mode of vaccination effectively bypasses the immunosuppressive effects of maternally-derived serum antibodies, which typically are found in the very young.

In all subjects, the precise amount of the composition administered and the timing and repetition of administration will be determined by various factors, including the nature of the composition, the patient's state of health and weight, the mode of administration, the nature of the formulation, etc. For polypeptide compositions, generally dosage levels of 0.001 to 20 mg/kg of body weight daily are administered to mammals. Generally, for intravenous injection or infusion, dosage may be lower.

Dosages for live virus will generally range from about 10³ to about 10⁶ plaque forming units (“PFU”) or more of virus per patient, more commonly from about 10⁴ to 10⁵ PFU virus per patient. In some embodiments, about 10⁵ to 10⁶ PFU per patient could be administered during infancy, such as between 1 and 6 months of age, and one or more additional booster doses could be given 2-6 months or more later. In another embodiment, young infants could be given a dose of about 10⁵ to 10⁶ PFU per patient at approximately 2, 4, and 6 months of age, which is the recommended time of administration of a number of other childhood vaccines. In yet another embodiment, an additional booster dose could be administered at approximately 10-15 months of age. In any event, the vaccine formulations should provide a quantity of virus sufficient to effectively stimulate or induce an anti-pneumoviruse immune response (an “effective amount”).

Any of the compositions can be administered as part of vaccine regime including 1, 2, 3, 4, 5, or more administrations of the disclosed compositions, 1, 2, 3, 4, 5, 5, 6, or 7 days, weeks, or months apart. In some embodiments, the vaccine regime includes a prime and boost, or a prime, a first boost, and a second boost. In a specific, non-limiting embodiment the, regime is a prime-boost regime, 3 or 4 weeks apart.

The immune responses can be characterized by a variety of methods. These include taking samples of nasal washes or sera for analysis of pneumoviruses-specific antibodies, which can be detected by tests including, but not limited to, complement fixation, plaque neutralization, enzyme-linked immunosorbent assay, luciferase-immunoprecipitation assay, and flow cytometry. In addition, immune responses can be detected by assay of cytokines in nasal washes or sera, ELISPOT of immune cells from either source, quantitative RT-PCR or microarray analysis of nasal wash or serum samples, and restimulation of immune cells from nasal washes or serum by re-exposure to viral antigen in vitro and analysis for the production or display of cytokines, surface markers, or other immune correlates measures by flow cytometry or for cytotoxic activity against indicator target cells displaying pneumovirus antigens. In some embodiments, testing including use of more or more of the exemplary assays of the experiments provided below.

Due to some patients having been previously exposed to pneumoviruses, tests may be performed before and after treatment with a disclosed composition, wherein an increased or induced immune response is evident by an increase or improvement after treatment relative to before treatment.

In some embodiments, individuals are also monitored for signs and symptoms of upper respiratory illness stemming from the treatment, particularly where the treatment includes administration of live virus.

In some embodiments, the subjects, which may be neonates, infants, children, adolescents, adults including or excluding the elderly, or any combination thereof, are given multiple doses of the composition to elicit sufficient levels of immunity. For neonates and infants, administration may begin within the first month of life, and continue at intervals throughout childhood, such as at two months, four months, six months, one year and two years, as necessary to maintain sufficient levels of protection against natural pneumovirus infection. In other embodiments, adults who are particularly susceptible to repeated or serious pneumoviruses infection, such as, for example, health care workers, day care workers, family members of young children, the elderly, individuals with compromised cardiopulmonary function, etc. are given multiple administrations to establish and/or maintain protective immune responses. Levels of induced immunity can be monitored by measuring amounts of neutralizing secretory and serum antibodies, and dosages adjusted and/or administrations repeated as necessary to maintain desired levels of protection.

Further, different compositions may be indicated for administration to different recipient groups. For example, an engineered RSV strain expressing a cytokine or an additional protein rich in T cell epitopes may be particularly advantageous for adults rather than for infants.

Any of the disclosed compositions can be combined with other conventional pneumovirus vaccination compositions and methods. For example, viruses of the other subgroup or strains of pneumoviruses can be combined withe disclosed compositions and methods to increase or expand protection against multiple subgroups or strains, or selected gene segments encoding, e.g., protective epitopes of these strains, which may also be engineered into a recombinant virus as described herein. In such embodiments, the different compositions can be in the same or different admixtures and administered simultaneously or present in separate preparations and administered separately.

The disclosed compositions may elicit production of an immune response that may be protective against, or reduce the magnitude of serious lower respiratory tract disease, such as pneumonia and bronchiolitis when the individual is subsequently infected with a wild-type or otherwise naturally occurring pneumovirus such as RSV and/or hMPV. For example, in some embodiments, the naturally circulating virus is still capable of causing infection, particularly in the upper respiratory tract, however, rhinitis and/or resistance to subsequent infection by wild-type virus are reduced following treatment with the disclosed compositions. Following treatment there may be detectable levels of host engendered serum and, in some instances, secretory antibodies which are capable of neutralizing homologous (of the same subgroup) wild-type virus in vitro and in vivo. In many instances the host antibodies will also neutralize wild-type virus of a different, non-vaccine subgroup.

### B. Viruses and Symptoms

The disclosed composition are believed to effective again multiple pneumoviruses. In some embodiments, the pneumoviruses include RSV, hMPV, or both.

Respiratory syncytial virus (RSV) and human metapneumovirus (hMPV) are two leading causes of severe respiratory infections in children, the elderly, and immunocompromised patients. RSV is divided into two antigenic subtypes, A and B, based on the reactivity of the F and G surface proteins to monoclonal antibodies. The subtypes tend to circulate simultaneously within local epidemics, although subtype A tends to be more prevalent. Generally, RSV subtype A (RSVA) is thought to be more virulent than RSV subtype B (RSVB), with higher viral loads and faster transmission time. To date, 16 RSVA and 22 RSVB clades (or strains) have been identified. Among RSVA, the GA1, GA2, GAS, and GA7 clades predominate; GA7 is found only in the United States. Among RSVB, the BA clade predominates worldwide. See, e.g., Griffiths, et al., Clinical Microbiology Reviews, 30 (1): 277-319 (2017). doi:10.1128/CMR.00010-16. RSV infection can present with a wide variety of signs and symptoms that range from mild upper respiratory tract infections (URTI) to severe and potentially life-threatening lower respiratory tract infections (LRTI) requiring hospitalization and mechanical ventilation. While RSV can cause respiratory tract infections in people of all ages and is among the most common childhood infections, its presentation often varies between age groups and immune status. Most childhood RSV infections are fairly self-limited with typical upper respiratory tract signs and symptoms, such as nasal congestion, runny nose, cough, and low-grade fever. Inflammation of the nasal mucosa (rhinitis) and throat (pharyngitis), as well as redness of the eyes (conjunctival infection), may be seen on exam. Approximately 15-50% of children will go on to develop more serious lower respiratory tracts infections, such as bronchiolitis, viral pneumonia, or croup. Infants are at the highest risk of disease progression. In very young infants under 6 weeks of age, and particularly in premature infants, signs of infection may be less specific. They may have minimal respiratory involvement. Instead, they may exhibit decreased activity, irritability, poor feeding, or breathing with difficulties. This can also be accompanied by apneic spells, or brief pauses in breathing. In adults, if present, symptoms are generally isolated to the upper respiratory tract: runny nose, sore throat, fever, and malaise. In the vast majority of cases, nasal congestion precedes the development of cough. In contrast to other upper respiratory infections, RSV is also more likely to cause new onset wheeze in adults. Infection may also be asymptomatic. While RSV very rarely causes severe disease in healthy adults, it can cause significant morbidity and mortality in the elderly and in those with underlying immune compromise or cardiopulmonary disease. Older adults have a similar presentation to younger adults but tend to have greater symptom severity with increased risk of lower respiratory tract involvement. In particular, the elderly are more likely to experience pneumonia, respiratory distress, and death.

In some embodiments, the disclosed compositions are used to treat or prevent an infection from an RSVA and/or RSVB or specific clade(s) (or strain(s)) thereof in subject in need thereof. The RSV can be, and typically is, a human RSV. However, other RSVs, such as bovine RSV, are also contemplated. In some embodiments, the compositions are used to treat or prevent one or more symptoms of an RSV infection.

HMPV is genetically similar to the avian metapneumoviruses A, B and in particular type C. Phylogenetic analysis of HMPV has demonstrated the existence of two main genetic lineages termed subtype A and B containing within them the subgroups A1/A2 and B1/B2 respectively. Genotyping based on sequences of the F and G genes showed that subtype B was associated with increased cough duration and increased general respiratory systems compared to hMPV-A. Mild symptoms of hMPV include cough, runny nose or nasal congestion, sore throat and fever. More severe illness, with wheezing, difficulty breathing, hoarseness, cough, pneumonia, and in adults, aggravation of asthma, also has been reported.

In some embodiments, the disclosed compositions are used to treat or prevent an hMPV subtype A1, A2, B1, and/or B2 infection in a subject in need thereof. In some embodiments, the compositions are used to treat or prevent one or more symptoms of a hMPV infection.

In some embodiments, the compositions are effective to treat or prevent another virus closely related to RSV and/or hMPV. Examples include, but are not limited to, an avian metapneumovirus, which have been divided into four subgroups—A, B, C and D.

In some embodiments, the disclosed compositions are administered in combination with other one more other, e.g., conventional approaches, particularly for vulnerable populations such as the young for example infants less than 24 months or less than 12; the old (e.g., the elderly), those as high risk for infection (e.g., healthcare workers), and/or those with sensitivities (e.g., chronic lung disease, congenital heart disease, congenital airway abnormality, neuromuscular disorder, cystic fibrosis, severely immunocompromised, and/or heart transplant candidates and recipients). For example, the compositions can be administered in combination with passive immunization, e.g., intravenous immunoglobin (IVIG), monoclonal antibody (MAb) that can be delivered through muscular injection, etc. A particular antibody is Palivizumab. Anti-viral co-therapies include, for example, ribavirin and presatovir. Other treatments include supportive care, anti-inflammatories, bronchodilators, and antibiotics.

The invention can be further understood by the following numbered paragraphs:

1. A chimeric polypeptide including one or more immunodominant epitopes of the fusion protein (F) of respiratory syncytial virus (RSV) and one or more immunodominant epitopes of the fusion protein (F) of human metapneumovirus (hMPV).

2. The chimeric polypeptide of paragraph 1, where the immunodominant epitopes from RSV are derived from the head of RSV F, the immunodominant epitope(s) from hMPV are derived from the stem of hMPV F, or a combination thereof.

3. The chimeric polypeptide of paragraphs 1 or 2, including the amino acid sequences of two or more, optionally all, antigenic sites selected from the group consisting of RSV site Ø, RSV site V, RSV site II, hMPV site III, hMPV site IV, and hMPV DS7 site optionally wherein the sites are defined according to FIG. 1B.

4. The chimeric polypeptide of paragraph 3, including two cleavage sites of RSV; one or DsCav1 mutations (S155C, S190F, V207L, and/or S290C with reference to SEQ ID NO:3); the fusion peptide of RSV F having the F2 N-terminus (residues 26-54 relative to SEQ ID NO:3) and the F1 C-terminus (residues 315-531 relative to SEQ ID NO:3) replaced by the homologous hMPV F sequences, with two junctions located on 32 and 07 strands; two glycosylation sites (RSV F-N70 relative to SEQ ID NO:3, hMPV F-N354 relative to SEQ ID NO:4 or hMPV F-N353 relative to SEQ ID NO:40); or a combination thereof.

5. The chimeric polypeptide of any one of paragraphs 1-4, including the amino acid sequence of SEQ ID NO:1, a fragment thereof, or a variant thereof optionally including at least 50, 60, 70, 75, 80, 85, 90, 95, or more percent sequence identity to SEQ ID NO:1.

6. A chimeric fusion protein including an antigenic domain including the chimeric polypeptide of any one of paragraphs 1-5 and one or more additional domains.

7. The chimeric fusion protein of paragraph 6 wherein the one or more additional domains are selected from a signal peptide sequence, a trimerization domain, a cleavage site, a purification tag or report sequence, and one or more linker sequences.

8. The chimeric fusion protein of paragraphs 6 or 7 including a trimerization domain, optionally wherein the trimerization domain is selected from GCN4-based isoleucine zipper (IZ), T4 bacteriophage fibritin foldon (Fd) trimerization domain, trimerization domain of collagen, the HIV gp41 trimerization domain, and the transmembrane domain and cytoplasmic tail of RSV or hMPV F protein, optionally wherein the trimerization domain includes the amino acid sequence of any one of SEQ ID NOS:19-26, 44, or 45.

9. The chimeric fusion protein of any one of paragraphs 6-8 including a signal peptide sequence, optionally wherein the peptide signal sequence is derived from RSV F, optionally wherein the signal peptide sequence includes the amino acid sequence of SEQ ID NO:5.

10. The chimeric fusion protein of any one of paragraphs 6-9 including a purification tag, optionally wherein the purification tag includes six consecutive histidine residues.

11. The chimeric fusion protein of any one of paragraphs 6-10, wherein when present, the orientation of the chimeric fusion protein domains is from N-terminus to C-terminus: signal peptide sequence-antigenic domain-trimerization domain-cleavage site-purification tag, optionally wherein one or more pairs of domains are separated by a linker sequence.

12. The chimeric fusion protein of any one of paragraphs 6-11 including the amino acid sequence of any one of SEQ ID NOS:2, or 41-43 or 46-47, or fragment, or variant thereof with at least 70% sequence identity thereto.

13. A nucleic acid encoding the chimeric polypeptide of any one of paragraphs 1-5 or the chimeric fusion protein of any one of paragraphs 6-12.

14. The nucleic acid of paragraph 13, wherein the nucleic acid is mRNA.

15. A vector including the nucleic acid of paragraph 13 operably linked to an expression control sequence.

16. The vector of paragraph 15, wherein the vector is a viral vector.

17. A recombinant virus genome or antigenome including the nucleic acid of paragraph 13, optionally wherein the recombinant virus is a recombinant RSV or recombinant hMPV, optionally wherein the recombinant virus is an attenuated virus.

18. A recombinant virus including the recombinant virus genome of paragraph 17.

19. A recombinant virus including the chimeric polypeptide of any one of paragraphs 1-5 or chimeric fusion protein of any one of paragraphs 6-12.

20. The recombinant virus of paragraph 19, wherein the virus is replication competent or replication incompetent optionally wherein the recombinant virus is a recombinant RSV or recombinant hMPV, optionally wherein the recombinant virus is an attenuated virus.

21. A method of making an chimeric polypeptide or fusion protein including expressing a nucleic acid encoding the chimeric polypeptide of any one of paragraphs 1-5 or the chimeric fusion protein of any one of paragraphs 6-12 in cells, optionally mammalian (e.g., human) or insect cells, and isolating the expressed chimeric polypeptide or fusion protein, optionally wherein the expressed chimeric polypeptide or fusion protein is a multimeric protein such as a trimer.

22. A macromolecule including multimerizations of a mature form of the chimeric polypeptide of any one of paragraphs 1-5 or the chimeric fusion protein of any one of paragraphs 6-12.

23. The macromolecule of paragraph 22, wherein the mature form of the chimeric polypeptide or fusion protein includes cleavage of chimeric polypeptide or fusion protein.

24. A pharmaceutical composition including (i) the chimeric polypeptide of any one of paragraphs 1-5, (ii) the chimeric fusion protein of any one of paragraphs 6-12 as monomer or a multimer optionally wherein the multimer is a trimer, (iii) the nucleic acid, vector, or viral genome or antigenome of any one of paragraphs 17-20, (iv) the recombinant virus of any one of paragraphs 18-20, (v) the expressed fusion protein made according to the method of paragraph 21, and/or (vi) the macromolecule of paragraphs 22 or 23, optionally packaged in delivery vehicle such as polymeric or liposomal nanoparticles.

25. The pharmaceutical composition of paragraph 24 further including an adjuvant.

26. A method of inducing or increasing an immune response in a subject in need thereof including administering the subject the pharmaceutical composition of paragraphs 24 or 25.

27. The method of paragraph 26, wherein the pharmaceutical composition is administered in an effective amount to increase immunity against one or more pneumoviruses in the subject.

28. The method of paragraph 27, wherein the one or more pneumoviruses include RSV, hMPV, or both.

29. The method of paragraph 28, wherein increased immunity includes increase neutralizing antibodies against the one or more pneumoviruses.

30. The method of paragraph 29, wherein the one or more pneumoviruses include RSV, hMPV, or both.

31. The method of any one of paragraphs 26-30, wherein the composition is administered prophylactically.

32. The method of any one of paragraphs 26-31, wherein administration reduces viral infection and/or one or more symptoms caused by viral infection in the subject.

33. The method of any one of paragraphs 26-32, wherein the subject is an infant, child, adult optionally elderly adult; optionally wherein the subject is a human.

34. A compound, composition, or method as described herein including but not limited to the text and drawings.

### EXAMPLES

### Example 1: RHMS-1 is a Pre-Fusion Trimer Antigen Candidate

### Materials and Methods

### Expression and Purification of Proteins

Plasmids encoding cDNAs of Pneumovirus proteins were synthesized (GenScript) and cloned into the pcDNA3.1+ vector (McLellan et al., Science (80-) 342:592-598 (2013), Huang et al., J Virol JVI0059321, Huang et al., PLoS Pathog 16:e1008942 (2020)). The stable cell line that expresses the hMPV B2 F protein was utilized as previously described (Huang et al., J Virol JVI0059321). The rest of the F proteins and monoclonal antibodies were transiently expressed in Expi293F cells. The proteins were harvested from the supernatant of cell cultures and purified by HisTrap Excel (for his-tagged proteins) or Protein G (for antibodies) columns (GE Healthcare Life Sciences). RHMS-1 (SEQ ID NO:2) (trimer), RSV A2 F (trimer), and trypsin-treated hMPV B2 F (monomer) were further purified by size exclusion chromatography on a Superdex S200, 16/600 column (GE Healthcare Life Sciences).

### Negative-Stain Electron Microscopy Analysis

Purified RHMS-1 (trimer) was applied on carbon-coated copper grids (5 μl of 10 μg/mL protein solution) for 3 minutes. The grid was washed in water twice then stained with Nano-W (Nanoprobes) for 1 minute. Negative-stain electron micrographs were acquired using a JEOL JEM1011 transmission electron microscope equipped with a high-contrast 2K-by-2K AMT midmount digital camera.

ELISA of RHMS-1 with mAbs or Human/Mouse Serum

384-well plates (Greiner Bio-One) were coated with 2 μg/ml of antigen in PBS overnight at 4° C. The plates were then washed once with water before blocking for 1 hour with blocking buffer. Primary mAbs (starting at 20 μg/ml and followed by 3-fold dilutions) or serial dilutions of human/mouse serum (starting with 1:50 and followed by 3-fold dilutions) were applied to wells for 1 hour after three washes with water. Plates were washed with water three times before applying 25 μl of secondary antibody (goat anti-human IgG Fc-Southern Biotech, 2048-04; goat anti-mouse IgG Fc-Southern Biotech, 1033-04) at a dilution of 1:4,000 in blocking buffer. After incubation for 1 hour, the plates were washed five times with 0.05% PBS-Tween-20, and 25 μl of a PNPP (p-nitrophenyl phosphate) substrate solution (1 mg/ml PNPP in 1 M Tris base) was added to each well. The plates were incubated at room temperature for 1 hour before reading the optical density at 405 nm (OD₄₀₅) on a BioTek plate reader. Data were analyzed in GraphPad Prism using a nonlinear regression curve fit and the log(agonist)-versus-response function to calculate the binding EC50 values. Mouse serum IgG titers were calculated from the highest dilution of a serum sample that produced OD405 readings of >0.3 above the background readings and were shown in a log 10 scale as previously described (Huang et al., J Virol JVI0059321).

### Results

The RHMS-1 protein (SEQ ID NO:2) was designed based on the pre-fusion structures of RSV F and hMPV F using ChimeraX (Pettersen et al., Protein Sci 30:70-82 (2021)). A model of RHMS-1 based on these structures is shown in FIG. 1A. The design of RHMS-1 maintains the signal peptide, two cleavage sites, DsCav1 mutations (S155C, S190F, V207L, S290C) (McLellan et al., Science (80-) 342:592-598 (2013)), and the fusion peptide of RSV F. Part of the F2 N-terminus (residues 26-54) and the F1 C-terminus (residues 315-531) was replaced by the homologous hMPV F sequences, with two junctions located on β2 and β7 strands. Two glycosylation sites (RSV F-N70, hMPV F-N353) were retained in RHMS-1. A GCN4 trimerization domain and a hexa-histidine tag were appended to the F1 C-terminus (FIG. 1B). Intact RSV F sites II, V, and Ø and hMPV F sites IV, DS7 were adopted from the original sequence. For antigenic site III, part of the cross-protomer site III of RHMS-1 is on the RSV head, but most of it is originated from hMPV F (FIG. 1C).

RHMS-1 was expressed in HEK293F cells and size exclusion chromatography (SEC) showed RHMS-1 was mainly expressed as a trimeric protein, but the size is slightly bigger than RSV F trimers (FIG. 2A). For hMPV F, trimers and monomers were observed after trypsin treatment as previously described (Huang et al., J Virol JVI0059321), but the size of trimeric hMPV F is smaller than RHMS-1 and RSV F, likely due to trypsinization (FIG. 2A). Like RSV F, RHMS-1 was deduced to be cleaved after expression, as the F2 domain was observed on the gel under reducing conditions (FIG. 2B), and the sizes of RHMS-1 and RSV F bands are consistent with the peaks shown in FIG. 2A. Negative-stain EM analysis demonstrated that majority of the particles are in pre-fusion conformation based on “ball-like” structures resembling pre-fusion RSV and hMPV F (Battles et al., Nat Commun 8:1-11 (2017), McLellan et al., Science (80-) 342:592-598 (2013)) (FIG. 2C), indicating the DsCav1 mutations work well in stabilizing the structure of RHMS-1.

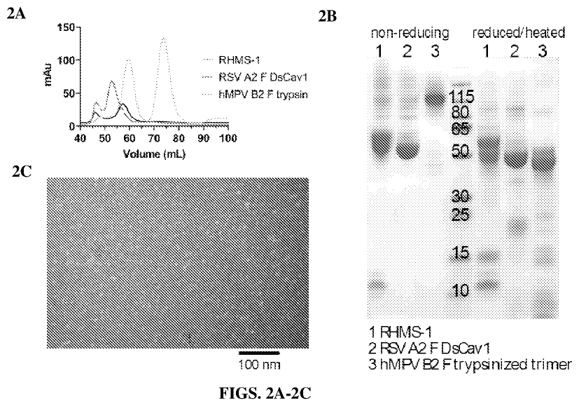

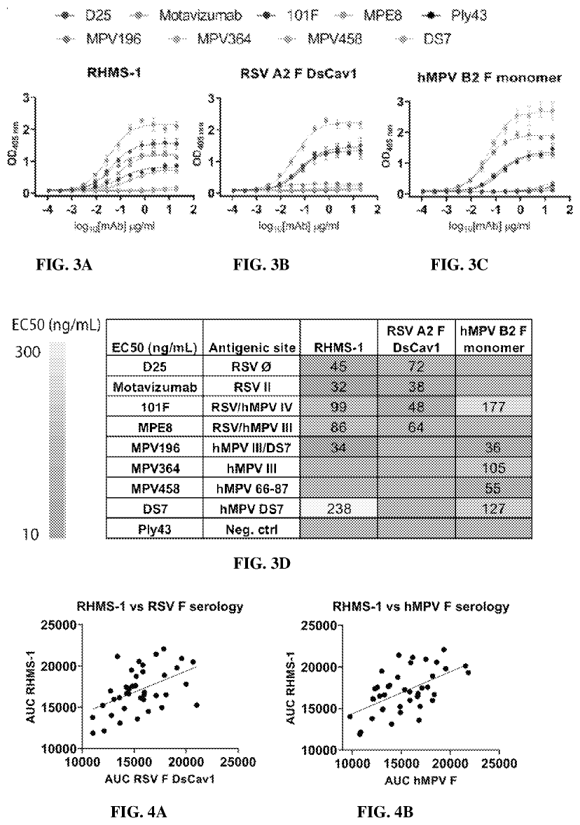

RHMS-1 shares immunological features of both RSV and hMPV F To determine if RHMS-1 retains the correct conformation of each antigenic site, mAbs specifically targeting these sites were tested for binding by ELISA (FIGS. 3A-3C). For both RSV F and RHMS-1, mAb D25 binds to site 0 (McLellan et al., Science (80-) 340:1113-1117 (2013)) and motavizumab binds to site II (Wu et al., J Mol Biol 368:652-665 (2007), Gilman et al., Nat Commun 10:1-13 (2019)) at similar EC50 values (FIG. 3D). For both hMPV F and RHMS-1, mAbs DS7 and MPV196 bind to the DS7 site (Williams et al., J Virol 81:8315-8324 (2007), Bar-Peled et al., J Virol 93 (2019)). mAb 101F binds to all three antigens on site IV (Más et al., PLoS Pathog 12:e1005859 (2016)) while mAb MPE8 binds to site III on RSV F and RHMS-1 (Corti et al., Nature 501:439-443 (2013)), but not monomeric hMPV F, likely due to the cross-protomer epitope that is only partially displayed on a single monomer. The binding site of mAb MPV364 partially overlaps with hMPV site III, but it was also predicted to interact with the head of hMPV F (Bar-Peled et al., J Virol 93 (2019)), and mAb MPV458 binds to the 66-87 peptide on the head of hMPV F (Huang et al., PLoS Pathog 16:e1008942 (2020)), therefore, both MPV364 and 458 do not bind to RHMS-1.

### Example 2: RHMS-1 can be Recognized by B Cells Pre-Exposed to RSV F or hMPV F

### Materials and Methods

### ELISA Screening Human PBMCs

As previously described (Bar-Peled et al., J Virol 93 (2019)), peripheral blood mononuclear cells (PBMCs) and plasma were isolated from human subject blood samples using CPT tubes (BD, 362753), and PBMCs were frozen in the liquid nitrogen vapor phase until further use. For serology screening, the plasma of 41 subjects were used for ELISA as described above. The IgG binding was quantified by area under the curve (AUC) values using GraphPad Prism. For PBMC screening, ten million PBMCs were mixed with 8 million previously frozen and gamma irradiated NIH 3T3 cells modified to express human CD40L, human interleukin-21 (IL-21), and human B-cell activating factor (BAFF) in 80 mL StemCell medium A (StemCell Technologies) containing 1 μg/mL of cyclosporine A (Millipore-Sigma). The mixture of cells was plated in four 96-well plates at 200 μl per well in StemCell medium A. After 6 days, culture supernatants were screened by ELISA for IgG binding to the RHMS-1 (trimer), RSV A2 DsCav1 F (trimer), and trypsin-treated hMPV B2 F (monomer). Each well is represented by a dot with the OD₄₀₅ against RSV/hMPV F as the x coordinate, and the OD₄₀₅ against RHMS-1 as the y coordinate.

### Results

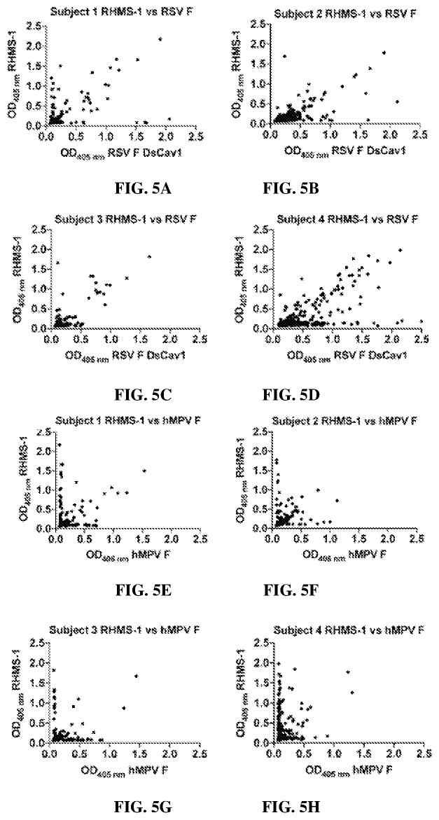

To verify if epitopes on RHMS-1 can be recognized by the human immune system in a similar manner comparing to RSV F or hMPV F, plasma IgG responses from 41 human subjects were screened against all 3 proteins by ELISA. Overall, positive correlations of serum IgG bindings were observed for both RSV F vs. RHMS-1 (FIG. 4A) and hMPV F vs. RHMS-1 (FIG. 4B), indicating epitopes are conserved between the native F proteins and the epitopes included on RHMS-1. The antibody responses at the cellular level were also tested by measuring the binding of supernatant from stimulated B cells in four subjects. The B cells in PBMCs were activated through coincubation with NIH 3T3 cells expressed human CD40L, human interleukin-21 (IL-21), and human B-cell activating factor (BAFF) to stimulate growth and IgG secretion to the culture supernatant as previously described (Bar-Peled et al., J Virol 93 (2019)). For all of the subjects tested, the majority of RSV F-positive B cells are also positive for RHMS-1 (FIGS. 5A-5D). The majority of human B cells target the head of the RSV F protein, which is retained in RHMS-1. Such correlations are still present for hMPV F (FIGS. 5E-5H), although the frequencies of hMPV F-positive B cells are generally lower than RSV F-positive B cells, therefore, populations of hMPV F negative, RHMS-1 positive B cells are seen close to the Y axes.

### Example 3: Vaccination with RHMS-1 Elicits Potent Neutralizing Antibodies and Cross-Protection in Mice

### Materials and Methods

Animal Immunization and hMPV/RSV Challenge.

BALB/c mice (6 to 8 weeks old; The Jackson Laboratory) were immunized in a prime-boost regimen with purified RHMS-1 (trimer), RSV A2 DsCav1 F (trimer), or trypsin-treated hMPV B2 F (monomer) (20 μg protein/mouse)+an equal volume of AddaS03 adjuvant via the subcutaneous route into the loose skin over the neck, while mice in control groups were immunized with PBS+AddaS03 adjuvant. Three weeks after prime, the mice were boosted with the same amount of the antigens+adjuvant. Three weeks after the boost, mice were bled and then intranasally challenged with RSV A2 (2.8×106 PFU per mouse) or hMPV TN/93-32 (3×105 PFU per mouse). Mice were sacrificed 5 days post-challenge, and lungs were collected and homogenized for virus titration as previously described (Huang et al., J Virol JVI0059321). Briefly, RSV challenged lung homogenates were plated on HEp-2 cells (EMEM+2% FBS) while hMPV challenged lung homogenates were plated on LLC-MK2 cells (EMEM+5 μg/ml trypsin-EDTA and 100 μg/ml CaCl2)) in 24 well plates. After 4-5 days, the cells were fixed with 10% neutral buffered formalin and the plaques of both viruses were immunostained with mAbs MPV364 (for hMPV) or 101F (for RSV). Plaques were counted by hand under a stereomicroscope.

Virus Neutralization Assays with Immunized Mice Serum

For serum neutralization assays, the serum of 4/8 mice were randomly picked from each group. Heat-inactivated mouse serum was serially diluted (starting at 1:25 and followed by 3-fold dilutions) and incubated 1:1 with a suspension of hMPV (CAN/97-83 and TN/93-32) or RSV (A2 and B) for 1 hour at room temperature. PBS was mixed with viruses as negative control. LLC-MK2 (for hMPV) or HEp-2 cells (for RSV) in 24-well plates were then inoculated with the serum-virus mixture (50 μL/well) for 1 hour and rocked at room temperature before adding the overlay. After 4-5 days, the plaques were stained as described above. The percent neutralization was calculated by (PFU in control wells—PFU in serum wells)/PFU in control wells×100%.

### Results

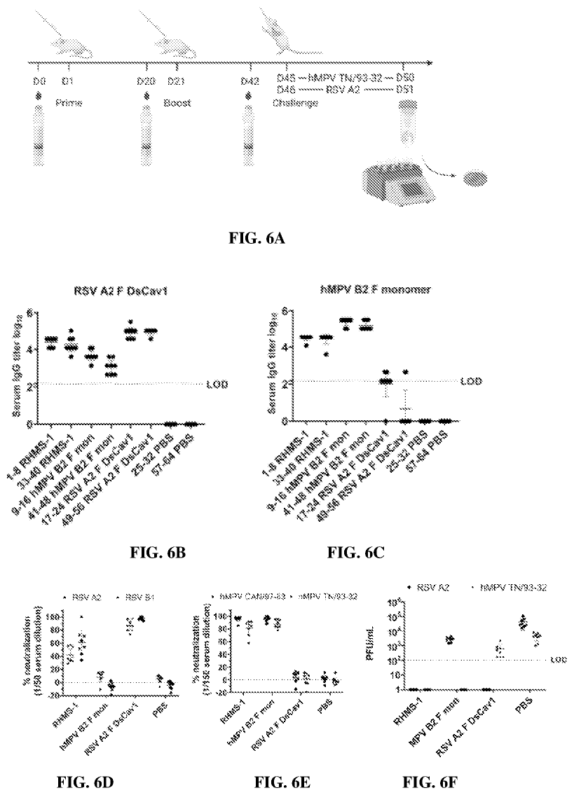

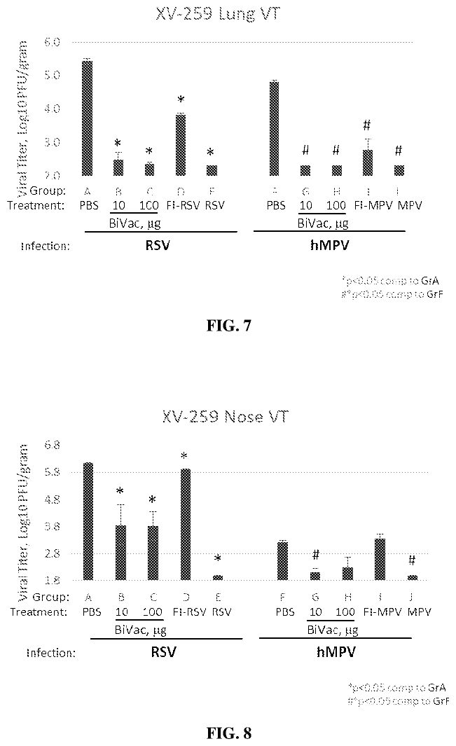

To evaluate the immunological properties of RHMS-1, it was tested as a vaccine in the mouse model. BALB/c mice were subcutaneously primed and boosted with 20 μg of RHMS-1, RSV F DsCav1, hMPV monomeric B2 F, or PBS in an emulsion formulated with AddaS03 adjuvant and then challenged with RSV or hMPV (FIG. 6A, Table 1).

TABLE 1

| Mice No. | Prime/Boost | Challenge |
|---|---|---|
| 1-8 | RHMS-1 | RSV A2 |
| 9-16 | hMPV B2 F monomer | |
| 17-24 | RSV A2 F DsCav1 | |
| 25-32 | PBS | |
| 33-40 | RHMS-1 | hMPV |
| 41-48 | hMPV B2 F monomer | TN/93-32 |
| 49-56 | RSV A2 F DsCav1 | |
| 57-64 | PBS | |

All RHMS-1 vaccinated mice showed serum IgG binding titers against both RSV F DsCav1 and hMPV monomeric B2 F proteins (FIG. 6B, 6C). Representative viruses from each RSV subgroup and each hMPV genotype were neutralized by RHMS-1 immunized mouse serum 3 weeks after the boost (FIG. 6D & 6E). RSV F DsCav1 immunization failed to induce IgG that cross-recognize hMPV monomeric B2 F, while hMPV monomeric B2 F immunized mice showed moderate binding, but non-neutralizing IgG against RSV. Three weeks after the boost, mice were intranasally challenged RSV A2 or hMPV TN/93-32. The virus titers in the lung homogenate were determined 5 days post challenge. Vaccination with RHMS-1 completely protected the mice from challenges of both viruses, while RSV A2 F DsCav1 and hMPV B2 F monomer vaccinated groups protected mice only against the autologous virus (FIG. 6F).

Multiple protein engineering strategies have been investigated to generate cross-protective or epitope-based antigens against RSV F or hMPV F. Previous attempts at grafted a single antigenic site on RSV F or hMPV F from one to another induced cross-neutralizing antibodies but very limited protection against the heterologous virus challenge, likely due to the epitopes on the backbone still dominate the immune responses (Olmedillas et al., EMBO Mol Med 10:175-187 (2018)). RHMS-1 contains multiple immunodominant epitopes of both RSV F and hMPV F in relatively equal proportions, including at least three RSV F-specific and three MPV F-specific antigenic sites. This may lead to more balanced immune responses against both RSV F and hMPV F, and maybe less likely to drive escape mutations focused on a single epitope. After prime and boost, RHMS-1 induced comparable levels of hMPV F/RSV F-specific serum IgG titers. Although the serum neutralization against RSV is not as potent as that against hMPV, RHMS-1 immunization completely protected the mice from both RSV and hMPV challenges, supporting the conclusion that RHMS-1 is a promising antigen that can be used as a vaccine to induce cross-neutralizing and cross-protecting antibodies against RSV and hMPV.

ELISA screening data showed that the human subjects tested had pre-existing immunity against both RSV F and hMPV F. Interestingly, subjects had an overall higher frequency of RSV F-specific B cells than hMPV F-specific B cells, which is likely due to the higher prevalence of RSV than hMPV. Since initial exposures to an antigen can influence subsequent immune responses against similar antigens, termed original antigenic sin (Francis, Proc Am Philos Soc, 104:572-578 (1960)), pre-existing immunity to RSV or hMPV may affect the efficacy of RHMS-1 or other RSV and hMPV F-based vaccine candidates.

Previous studies have shown that mice immunized with either pre-fusion RSV F or post-fusion hMPV F did not induce significant cross-neutralization antibodies (Más et al., PLoS Pathog 12:e1005859 (2016)). Similar results were observed in this study with pre-fusion RSV F and monomeric hMPV F. The serum of 9 out of 16 mice immunized with RSV F+AddaS03 showed little binding to monomeric hMPV F just above the detection limit, while all of the monomeric hMPV F+AddaS03 immunized mice serum had moderate binding to RSV F. However, the serum of both groups failed to cross-neutralize the viruses in vitro (FIG. 6B-6E). Interestingly, the lung virus counts in RSV F immunized/hMPV challenged and hMPV F immunized/RSV challenged groups are reduced ˜10 fold compared to mock immunized/hMPV challenged and mock immunized/RSV challenged groups respectively (FIG. 6F), indicating poorly and non-neutralizing, cross-reactive antibodies could play a role in limiting virus replication in the lungs of mice, possibly through Fc-mediated effector functions.

In summary, a chimeric RSV F and hMPV F protein, RHMS-1 was developed and characterized. This is believed to be the first immunogen that elicits potent and balanced protective immune responses against both RSV and hMPV. Moreover, RHMS-1 can be readily applied to both traditional and innovative vaccine delivery platforms like viral vectors, VLPs, nanoparticles, and mRNA, and may serve as a platform for a safe and effective universal Pneumovirus vaccine.

### Example 4: In-Vivo Assessment of the Safety, Efficacy, and Immunogenicity of a Bivalent hMPV-RSV Vaccine Against hMPV and RSV/A2 Challenge in a Cotton Rat Model

### Materials and Methods

### Animals

Fifty (50) inbred, 6-8 weeks old, Sigmodon hispidus female and male cotton rats (Sigmovir Biosystems, Inc., Rockville MD) were maintained and handled under veterinary supervision in accordance with the National Institutes of Health guidelines and Sigmovir Institutional Animal Care and Use Committee's approved animal study protocol (IACUC Protocol #15). Each group of 5 animals included 3 females (the first three animals in each group) and 2 males (the last 2 animals in each group). Cotton rats were housed in clear polycarbonate cages and provided with standard rodent chow (Harlan #7004) and tap water ad lib.

### Virus

Respiratory Syncytial Virus strain A/A2 (RSV A/A2) (ATCC, Manassas, VA) was propagated in HEp-2 cells after serial plaque-purification to reduce defective-interfering particles. A pool of virus designated as hRSV A/A2 (Lot #092215 SSM) containing approximately 3.0×10⁸ pfu/mL in sucrose stabilizing media was used in this in vivo experiment. Human Metapneumovirus strain A2 (obtained from Vanderbilt University) was propagated in LLC-MK2 cells. A pool of virus, designated as hMPV/A2 (Lot #030116 SSM), and containing approximately 3×10⁶ pfu/ml in sucrose stabilizing media was used. Both stocks of viruses were stored at −80° C. and were characterized in vivo using the cotton rat model, and validated for upper and lower respiratory tract replication.

### Summary of Methods

Animal ID: Eartag
Bleeding: Retro-orbital sinus bleed
Route of infection/priming: Intranasal inoculation (IN)
Route of treatment: Intramuscular (IM)

### Experimental Design and Procedures

On Day 0, 50 female and male young adult cotton rats (6-8 weeks of age) were divided into 10 groups of 5 animals each (3 females, 2 males). The animals were pre-bled for serum collection and ear tagged. Then, the animals were immunized or infected with 0.1 ml of preparation as indicated in Table 2 below (IN=intranasally; IM=intramuscularly). The vaccine protein concentration was 1.6 mg/mL. The 100 μg group required 63 μL of protein mixed 1:1 with adjuvant (AddaSO3 (Invitrogen)), for a total of 126 μL per animal per vaccination. For example, two tubes at 1.6 mg/mL in a volume of 800 μL per tube.

TABLE 2
Summary of Immunization or Infection

| | | | | TA Dose | | Immunization | | Virus | Day of |
|---|---|---|---|---|---|---|---|---|---|
| Group | N | Day 0 | Day 28 | (per rat) | | days | Route | Challenge | harvest |
| A | 5 | PBS (Mock) | PBS (Mock) | n/a | | 0, 28 | IM | RSV | 5 |
| B | 5 | [Bivalent Vacc] | [Bivalent Vacc] | 10 | μg | 0, 28 | IM | RSV | 5 |
| C | 5 | [Bivalent Vacc] | [Bivalent Vacc] | 100 | μg | 0, 28 | IM | RSV | 5 |
| D | 5 | FI-RSV | FI-RSV | 1:100 | | 0, 28 | IM/IM | RSV | 5 |
| | | Lot#100 | Lot#100 | in PBS | | | | | |
| E | 5 | RSV/A2 Live | — | 5.0 | | 0 only | IN | RSV | 5 |
| | | | | Log10 | | | | | |
| F | 5 | PBS (Mock) | PBS (Mock) | n/a | | 0, 28 | IM | hMPV | 5 |
| G | 5 | [Bivalent Vacc] | [Bivalent Vacc] | 10 | μg | 0, 28 | IM | hMPV | 5 |
| H | 5 | [Bivalent Vacc] | [Bivalent Vacc] | 100 | μg | 0, 28 | IM | hMPV | 5 |

| | | | | | | | | |
|---|---|---|---|---|---|---|---|---|
| I | 5 | FI-hMPV | FI-hMPV | 1:25 | 0, 28 | IM/IM | hMPV | 5 |
| | | | | in PBS | | | | |
| J | 5 | hMPV/A2 Live | — | 5.0 | 0 only | IN | hMPV | 5 |
| | | | | Log10 | | | | |

On Day 28, all animals were eye bled for serum collection. Animals in Groups A through D and Groups F through I were boosted with 0.1 ml of the preparation as indicated in Table 2 above. On Day 49, all animals were eye-bled for serum collection. Animals in groups A through E were challenge intranasally (IN) with 0.1 ml of RSV/A2 (Lot #092215 SSM) at 10⁵ PFU per animal. Animals in Groups F thru J were challenge IN with 0.1 ml of hMPV/A2 (Lot #030116 SSM) at 10⁵ PFU per animal. Back titration was performed to confirm the dose of virus used. On Day 54, all animals were sacrificed. The nasal tissue was harvested and homogenized in 3 ml of HBSS supplemented with 10% SPG for viral titrations. The lung was harvested en bloc and trisected for viral titration (left section; homogenized in 3 ml of HBSS supplemented with 10% SPG), histopathology (right section; inflated with 10% neutral buffered formalin), and qPCR (lingular lobe; flash frozen in liquid nitrogen). A summary of the samples collected and endpoint assays are detailed in Table 3 and Table 4 respectively.

TABLE 3
Summary of Sample Collections

| Sample Collections | Day(s) of collection | # of Sets |
|---|---|---|
| Lung and nose homogenates | 54 | 2 |
| Lung histology | 54 | 1 |
| Serum | 0, 28, 49 | 2 |
| Lung total RNA | 54 | 1 |

TABLE 4
Summary of Endpoint Assays

| Endpoint Assays | Samples | Storage |
|---|---|---|
| RSV viral titrations | Lung homogenates | −80° C. |
| (Groups A-E) | Nose homogenates | |
| hMPV viral titrations | Lung homogenates | −80° C. |
| (Groups F-J) | Nose homogenates | |
| RSV Neutralizing Ab (RSV/A2) | Serum | −20° C. |
| (d 0, 28, and 49) | | |
| RSV/A2 serum IgG ELISA | Serum | −20° C. |
| (d 0, 28, 49) (1-dln format) | | |
| hMPV Neutralizing Ab (hMPV/A2) | Serum | −20° C. |
| (d 0, 28, and 49) | | |
| hMPV serum IgG ELISA | Serum | −20° C. |
| (d 0, 28, 49) (1-dln format) | | |
| Pulmonary Histopathology | H&E lung slides | RT |
| qPCR | Lung RNA | −80° C. |
| RSV/A2 NS-1 gene (Groups A-E) | | |
| hMPV/A2 L gene (Groups F-J) | | |
| IL-4, IL-5, IFN-γ | | |

### RSV Lung and Nose Viral Titration

Lung and nose homogenates were clarified by centrifugation and diluted in Eagle's Minimum Essential Medium (EMEM). Confluent HEp-2 monolayers are infected in duplicates with diluted homogenates in 24 well plates. After one hour incubation at 37° C. in a 5% CO2 incubator, the wells were overlayed with 0.75% Methylcellulose medium. After 4 days of incubation, the overlay was removed, and the cells were fixed with 0.1% crystal violet stain for one hour, then rinsed and air dried. Plaques were counted and virus titer was expressed as plaque forming units per gram of tissue. Viral titers are calculated as geometric mean±standard error for all animals in a group at a given time.

hMPV/A2 Lung and Nose Viral Titration

Lung and nose homogenates were clarified by centrifugation and diluted in EMEM. Confluent LLC-MK-2 monolayers were infected in duplicates with diluted homogenates in 24 well plates. After one hour incubation at 37° C. in a 5% CO₂ incubator, the wells were overlayed with 0.75% methylcellulose medium. After 7 days of incubation, the overlays were removed, the cells were mixed for one hour and air-dried for immuno-staining. Upon blocking the wells with 1% BSA in PBS, mouse anti-hMPV-N-protein antibody at a 1:1,000 dilution in 1% BSA was added to each well, followed by washes and incubation with HRP-conjugated Rabbit anti-mouse IgG diluted 1:1,000 in 1% BSA. AEC Chromogen detection solution was added to each well and incubated at room temperature for 2 hours. Visible plaques are counted and virus titers were expressed as plaque forming units per gram of tissue. Viral titers were calculated as geometric mean±standard error for all animals in a group at a given time.

### RSV Neutralizing Antibody Assay (60% Reduction)

Heat inactivated sera samples were diluted 1:10 with EMEM and serially diluted further 1:4. Diluted sera samples were incubated with RSV (25-50 PFU) for 1 hour at room temperature and inoculated in duplicates onto confluent HEp-2 monolayers in 24 well plates. After one hour incubation at 37° C. in a 5% CO₂ incubator, the wells were overlayed with 0.75% Methylcellulose medium. After 4 days of incubation the overlay was removed and the cells were fixed with 0.1% crystal violet stain for one hour, then rinsed and air dried. The corresponding reciprocal neutralizing antibody titers were determined at the 60% reduction end-point of the virus control using the statistics program “plqrd.manual.entry”. The geometric means±standard error for all animals in a group at a given time were calculated.

### HMPV Neutralizing Antibody Assay (60% PRNT)

Heat inactivated sera samples were diluted 1:10 with EMEM and serially diluted further 1:4. Diluted serum samples were incubated with hMPV/A2 (25-50 PFU) for 1 hour at room temperature and inoculated in duplicates onto confluent LLC-ML2 monolayers in 24 well plates. After one hour incubation at 37° C. in a 5% CO₂ incubator, the wells were overlayed with 0.75% Methylcellulose medium. After 7 days of incubation, the overlays were removed, the cells were fixed for one hour, and air-dried for immuno-staining. Upon blocking the wells with 1% BSA in PBS, mouse anti-hMPV N protein at a 1:1,000 dilution in 1% BSA was added to each well, followed by washes and incubation with HRP conjugated Rabbit anti-mouse IgG diluted at 1:1,000 in 1% BSA. AEC Chromogen detection solution was added to each well and incubated at room temperature for 2 hours. Visible plaques were counted, and virus titers were expressed as plaque forming units per gram of tissue. Viral titers are calculated as geometric mean±standard error for all animals in a group at a given time.

### RSV Binding IgG Antibodies (ELISA)

F protein extracted from RSV-infected HEp-2 cells was diluted and coated onto 96 well ELISA plate overnight. The coating antigen was decanted, and the plate was incubated in blocking solution for one hour at room temperature and subsequently washed. Diluted sera (1:500 in duplicates) along with the positive and negative controls were added to the wells and incubated at room temperature for one hour. After washing the plates, Chicken-anti-CR IgG-HRP (1:20,000) was added to all the wells and incubated for 30 minutes at room temperature. For development of the assay, TMB (3,3′,5,5′-Tetramethylbenzidine) substrate was added to all the wells and incubated at room temperature for 15 minutes. TMB-Stop solution was added to all the wells and optical density at 450 nm was recorded. Geometric mean of the optical density (OD450) was measured for all duplicate sera samples.

### HMPV Binding IgG Antibodies (ELISA)

Methanol/Acetone fixed hMPV-infected (7 days post infection) LLC-MK2 cells were used as coating antigens in a 96 well ELISA plate format. The plates were incubated in blocking solution for one hour at room temperature and subsequently washed. Diluted sera (1:500 in duplicates) along with the positive and negative controls were added to the wells and incubated at room temperature for one hour. After washing the plates, Chicken-anti-CR IgG-HRP (1:20,000) was added to all the wells and incubated for 30 minutes at room temperature. For development of the assay, TMB substrate was added to all the wells and incubated at room temperature for 15 minutes. TMB-Stop solution was added to all the wells and optical density at 450 nm was recorded. Geometric mean of the optical density (OD450) was measured for all duplicate sera samples.

### Pulmonary Histopathology

Lungs were dissected and inflated with 10% neutral buffered formalin to their normal volume, and then immersed in the same fixative solution. Following fixation, the lungs were embedded in paraffin, sectioned and stained with hematoxylin and eosin (H&E). Four parameters of pulmonary inflammation were evaluated: peri bronchiolitis (inflammatory cell infiltration around the bronchioles), perivasculitis (inflammatory cell infiltration around the small blood vessels), interstitial pneumonia (inflammatory cell infiltration and thickening of alveolar walls), and alveolitis (cells within the alveolar spaces). Slides were scored blind on a 0-4 severity scale. The scores were subsequently converted to a 0-100% histopathology scale.

### Real-Time PCR

Total RNA was extracted from homogenized tissue or cells using the RNeasy purification kit (QIAGEN). One g of total RNA was used to prepare cDNA using Super Script II RT (Invitrogen) and oligo dT primer (1 μl, Invitrogen). For the real-time PCR reactions, the Bio-Rad iQTM SYBR Green Supermix was used in a final volume of 25 μl, with final primer concentrations of 0.5 M. Reactions were set up in duplicates in 96-well trays. Amplifications are performed on a Bio-Rad iCycler for 1 cycle of 95° C. for 3 minutes, followed by 40 cycles of 95° C. for 10 seconds (s), 60° C. for 10 seconds, and 72° C. for 15 seconds. The baseline cycles and cycle threshold (Ct) were calculated by the iQ5 software in the PCR Base Line Subtracted Curve Fit mode. Relative quantitation of DNA was applied to all samples. The standard curves were developed using serially diluted cDNA sample most enriched in the transcript of interest (e.g., lungs from post-RSV or -hMPV infection of FI-RSV- or FI-hMPV-immunized animals, respectively). The Ct values were plotted against log₁₀ cDNA dilution factor. These curves were used to convert the Ct values obtained for different samples to relative expression units. These relative expression units were then normalized to the level of β-actin mRNA (“housekeeping gene”) expressed in the corresponding sample. For animal studies, mRNA levels were expressed as the geometric mean±SEM for all animals in a group at a given time.

### Results

### Lung Viral Titers

RSV: RSV A/A2 load in the lungs of cotton rats in Groups A-E was evaluated 5 days after intranasal RSV challenge. RSV-infected animals mock-treated with PBS intramuscularly (i.m.) (Group A) exhibited a titer of 5.43 Log₁₀ PFU/g virus in the lungs (FIG. 7). Animals immunized with a bivalent vaccine (BiVac, also referred to herein as RHMS-1, e.g., made by expression of the polypeptide of SEQ ID NO:2) at doses of 10 μg or 100 μg (Groups B and C, respectively) had significantly lower pulmonary RSV load (2.49 and 2.36 Log 10 PFU/g, respectively; FIG. 7). No virus was detectable in 3/5 animals immunized with 10 μg (Group B) and 4/5 animals immunized with 100 μg (Group C) of BiVac (FIG. 7). RSV titer in animals immunized with FI-RSV (Group D) was 3.83 Log₁₀ PFU/g, and no virus was detected in animals immunized via intranasal RSV infection (Group E; FIG. 7).

hMPV: hMPV load in the lungs of cotton rats in Groups F-J was evaluated 5 days after intranasal hMPV challenge. hMPV-infected animals treated with PBS i.m. (Group F) exhibited a titer of 4.81 Log₁₀ PFU/g in the lungs (FIG. 7). Animals immunized with BiVac at doses of 10 μg or 100 μg (Groups G and H, respectively) did not have any detectable hMPV in the lungs (FIG. 7). hMPV titer in animals immunized with FI-hMPV (Group I) was 2.78 Log 10 PFU/g, and no virus was detected in animals immunized via intranasal hMPV infection (Group J; FIG. 7).

### Nose Viral Titers

RSV: RSV A/A2 load in the noses of cotton rats in Groups A-E was evaluated 5 days following intranasal RSV challenge. RSV-infected animals mock-treated with PBS i.m. (Group A) had a titer of 6.14 Log₁₀ PFU/g virus in the nose (FIG. 8). RSV load in the noses of animals immunized with BiVac at 10 μg or 100 μg (Groups B and C, respectively) was reduced to 3.86 and 3.82 Log₁₀ PFU/g, respectively (FIG. 8). FI-RSV-immunized animals (Group D) had 5.93 Log₁₀ PFU/g in the nose and no virus was detected in animals immunized via intranasal RSV infection (Group E; FIG. 8).

hMPV: hMPV load in the noses of cotton rats in Groups F-J was evaluated 5 days after intranasal hMPV challenge. hMPV-infected animals mock-treated with PBS i.m. (Group F) exhibited a titer of 3.22 Log₁₀ PFU/g virus in the nose (FIG. 8). Nasal load in animals immunized with BiVac at doses of 10 μg or 100 μg (Groups G and H, respectively) was reduced to 2.12 and 2.29 Log₁₀ PFU/g, respectively, with the difference reaching statistical significance for the 10 μg vaccine dose (Group G; FIG. 8). FI-hMPV-immunized animals (Group I) had 3.35 Log₁₀ PFU/g in the nose and no virus was detected in animals immunized via intranasal hMPV infection (Group J; FIG. 8).

### Lung Histopathology

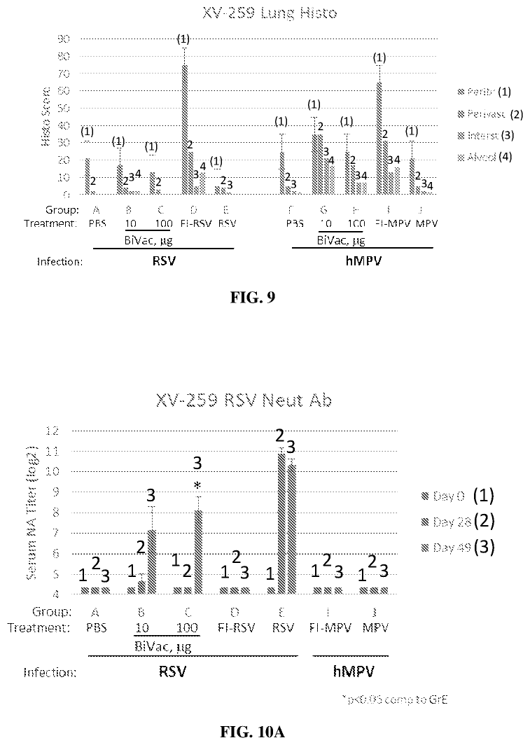

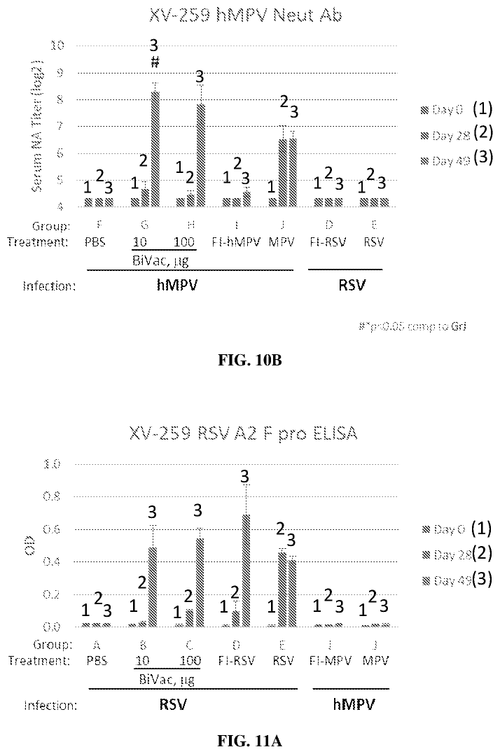

Pulmonary histopathology was evaluated in all animals 5 days after viral challenge. Among RSV-infected animals (Groups A-E), the highest level of peri bronchiolitis, perivasculitis, and alveolitis was detected in animals immunized with FI-RSV (Group D; FIG. 9). Pulmonary pathology in animals immunized with BiVac at doses of 10 μg or 100 μg (Groups B and C, respectively) did not exceed that seen in mock-immunized animals (Group A) or in animals immunized via intranasal RSV infection (Group E) (FIG. 9).

Among hMPV-infected animals (Groups F-J), the highest level of peri bronchiolitis was detected in animals immunized with FI-hMPV (Group I; FIG. 9). Peri bronchiolitis was lower in animals immunized with BiVac at doses of 10 μg or 100 μg (Groups B and C, respectively), and comparable to that detected in mock-immunized animals (Group F) or animals immunized via intranasal hMPV infection (Group J; FIG. 9). Animals immunized with BiVac at dose of 10 μg (Group B) showed elevated perivasculitis, interstitial inflammation, and alveolitis compared to mock-immunized animals (Group F) or animals immunized via intranasal hMPV infection (Group J; FIG. 9). These parameters were elevated to a smaller extent in animals immunized with BiVac at a dose of 100 μg (Group H).

### Serum Neutralizing Antibodies

Serum neutralizing antibodies against RSV A/A2 and against hMPV were measured in animals immunized with BiVac, infected with RSV or hMPV, immunized with FI-RSV or FI-hMPV, and control naïve animals.

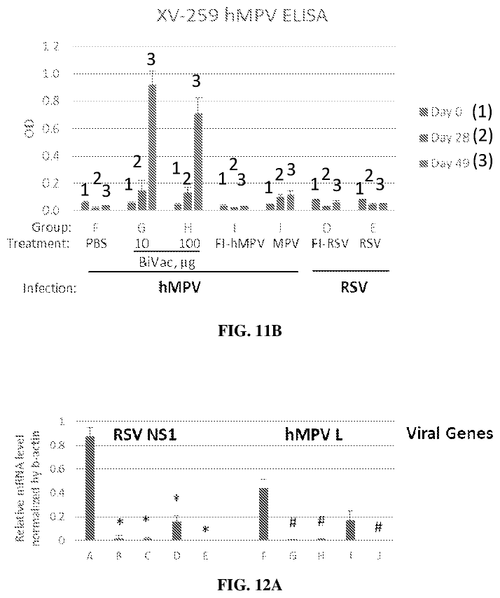

RSV neutralizing antibodies: Neutralizing antibodies (NA) against RSV were highest in the serum of animals previously infected with RSV (Group E; FIG. 10A). Levels of NA in RSV-infected animals were 10.88 log₂ on day 28 and 10.32 log₂ on day 49 post-infection (FIG. 10A).

Immunization with BiVac at 10 μg or 100 μg (Groups B or C, respectively) induced moderate levels of anti-RSV NA 3 weeks after boosting (Groups B and C, 7.15 and 8.14 log₂, respectively on day 49; FIG. 10A). After the first immunization (day 28 samples), NA antibodies against RSV were detected in one out of five animals vaccinated with 10 μg of BiVac (Group B) and in none of the animals vaccinated with 100 μg of BiVac (Group C; FIG. 10A). No cross-reactive neutralizing antibodies against RSV were detected in serum of animals infected with hMPV (Group J) or immunized with FI-hMPV (Group I; FIG. 10A).

hMPV neutralizing antibodies: Neutralizing antibodies against hMPV in the serum of animals previously infected with hMPV (Group J) were 6.54 log 2 on days 28 and 49 post-infection (FIG. 10B). Immunization and boosting with BiVac at 10 μg or 100 μg (Groups G or H, respectively), induced higher levels of hMPV NA (8.29 and 7.8 log₂, respectively, day 49 samples; FIG. 10B). After the first immunization (day 28 samples), NA antibodies against hMPV were detected in one out of 5 animals vaccinated with 10 μg or 100 μg of BiVac (Groups G or H, respectively; FIG. 10B). No cross-reactive neutralizing antibodies against hMPV were detected in serum of animals infected with RSV (Group E) or immunized with FI-RSV (Group D; FIG. 10B).

### Serum Binding IgG Antibodies

Serum binding IgG against RSV A/A2 F protein and against hMPV were measured in animals immunized with BiVac, infected with RSV or hMPV, immunized with FI-RSV or FI-hMPV, and control naïve animals.

RSV F protein IgG: The level of binding IgG against RSV F protein was highest in day 49 sera samples of animals immunized with 10 μg or 100 μg of BiVac (Groups B and C) and in FI-RSV-immunized animals (Group D; FIG. 11A). Anti-F IgG was slightly lower in day 28 and 49 samples of RSV-infected animals (Group E) (comparable level between the two days; FIG. 11A). Anti-F IgG was detectable at a much lower level in serum of animals immunized with the highest dose of BiVac (100 μg, Group C) and in FI-RSV-immunized animals (Group D; FIG. 11A). No cross-reactive IgG against RSV A/A2 F protein was detected in serum of animals infected with hMPV (Group J) or immunized with FI-hMPV (Group I; FIG. 11A).

hMPV IgG: The level of binding IgG against hMPV was highest in day 49 sera samples of animals immunized with 10 μg or 100 μg of BiVac (Groups G and H; FIG. 11B). A small increase in anti-hMPV IgG was detected in animals infected with hMPV (Group J; FIG. 11B). No binding IgG against hMPV was detected in animals vaccinated with FI-hMPV (FIG. 11B). No cross-reactive binding IgG against hMPV was detected in serum of animals infected with RSV (Group E) or immunized with FI-RSV (Group D; FIG. 11B).

qPCR Results

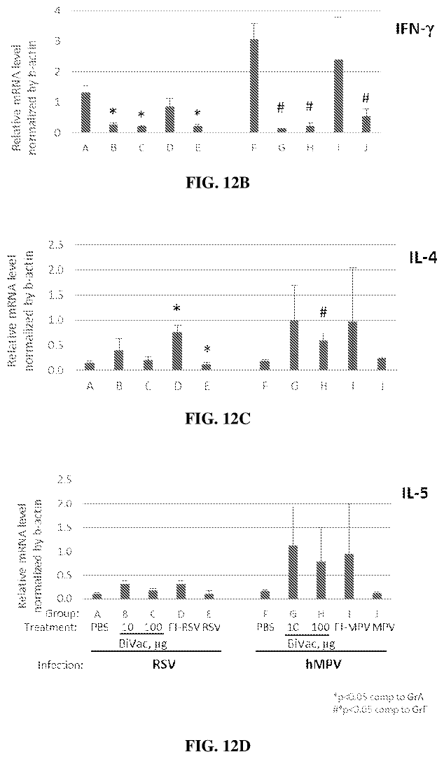

Expression of mRNA for RSV NS1 protein (FIG. 12A), hMPV L protein (FIG. 12A), IL-4 (FIG. 12C), and IFN-γ (FIG. 12B) was evaluated in lung samples collected on day 5. Expression of viral genes was significantly reduced in animals vaccinated with either dose of BiVac and infected with RSV (Groups B, C and E) or hMPV (Groups G, H and J) compared to animals with primary RSV (Group A) or primary hMPV (Group F) infections, respectively (FIG. 12A). The same result was found for pulmonary IFN-γ mRNA expression (FIG. 12B). Pulmonary expression of IL-4 was elevated in RSV-infected animals immunized with FI-RSV (Group D, p<0.05), slightly elevated in RSV-infected animals immunized with 10 μg of BiVac (Group B, no significance), and in hMPV-infected groups immunized with FI-hMPV or BiVac (Groups G, H, and I. p<0.05 for Group H; FIG. 12C). IL-5 mRNA levels were elevated in hMPV-infected groups immunized with FI-hMPV or BiVac (Groups G, H, and I, no significance; FIG. 12D).

### Discussion

Safety, efficacy, and immunogenicity of a bivalent hMPV-RSV vaccine (BiVac) was evaluated in the cotton rat S. hispidus models of RSV A/A2 and hMPV challenge. Young female and male cotton rats were immunized with BiVac at doses of 10 or 100 μg, boosted 4 weeks later, and challenged after 3 more weeks with either RSV A/A2 or hMPV/A2 at an inoculum dose of 10⁵ PFU per animal. All animals were sacrificed on day 5 post-infection for analysis of viral load, pulmonary histopathology, lung mRNA gene expression, and serum antibodies. Primary and secondary RSV and hMPV infection controls were included, as well as control groups of animals immunized with formalin-inactivated RSV or hMPV and infected with the corresponding live virus.

BiVac was highly efficacious at protecting the lung against RSV and hMPV infections, inducing sterilizing or near-sterilizing immunity against hMPV and RSV at both doses. BiVac was also effective at protecting the noses against replication of RSV and hMPV at both doses, resulting in a reduction in nasal viral titers of ˜2 Log₁₀ PFU/g for RSV and ˜1 Log₁₀ PFU/g for hMPV. BiVac induced high levels of serum neutralizing and binding IgG antibodies against RSV and hMPV after the second (booster) immunization. Anti-hMPV neutralizing and binding IgG antibody response induced by BiVac (in either dose) surpassed corresponding antibody responses induced by primary hMPV infection. BiVac did not enhance pulmonary pathology after RSV infection in either dose tested. Pulmonary pathology after hMPV infection, however, was associated with increased perivasculitis, interstitial inflammation, and alveolitis in animals that received BiVac compared to animals with primary or secondary hMPV infections. This increase in pathology was less pronounced for the 100 μg BiVac dose than for the 10 μg dose. qPCR analysis confirmed antiviral efficacy of BiVac against RSV and hMPV and showed that BiVac reduced pulmonary IFN-γ mRNA expression in RSV- and hMPV-challenged animals and increased the expression of Th2-type cytokines in hMPV-challenged animals vaccinated with either dose of BiVac.

Overall, BiVac was effective in the cotton rat models of RSV and hMPV challenge. A sterilizing or near-sterilizing immunity in the lung, and a strong protection of the nose was achieved in BiVac-vaccinated animals. A strong neutralizing antibody response against RSV and hMPV was induced by immunization with BiVac, which in case of hMPV surpassed that of natural hMPV infection modeled by intranasal hMPV challenge of naïve animals. No enhanced pathology was seen in RSV-challenged animals immunized with BiVac. Some parameters of pulmonary inflammation and Th2-type cytokines were elevated in BiVac-vaccinated animals infected with hMPV. Increasing BiVac dose from 10 to 100 μg was associated with a safer histologic profile in hMPV-challenged animals.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A chimeric polypeptide comprising one or more immunodominant epitopes of the fusion protein (F) of respiratory syncytial virus (RSV) and one or more immunodominant epitopes of the fusion protein (F) of human metapneumovirus (hMPV).

2. The chimeric polypeptide of claim 1, where the immunodominant epitopes from RSV are derived from the head of RSV F, the immunodominant epitope(s) from hMPV are derived from the stem of hMPV F, or a combination thereof.

3. The chimeric polypeptide of claim 1, comprising the amino acid sequences of two or more, optionally all, antigenic sites selected from the group consisting of RSV site Ø, RSV site V, RSV site II, hMPV site III, hMPV site IV, and hMPV DS7 site.

4. The chimeric polypeptide of claim 3, comprising two cleavage sites of RSV; one or DsCav1 mutations (S155C, S190F, V207L, and/or S290C with reference to SEQ ID NO:3); the fusion peptide of RSV F having the F2 N-terminus (residues 26-54 relative to SEQ ID NO:3) and the F1 C-terminus (residues 315-531 relative to SEQ ID NO:3) replaced by the homologous hMPV F sequences, with two junctions located on β2 and β7 strands; two glycosylation sites (RSV F-N70 relative to SEQ ID NO:3, hMPV F-N354 relative to SEQ ID NO:4 or hMPV F-N353 relative to SEQ ID NO:40); or a combination thereof.

5. The chimeric polypeptide of claim 1, comprising the amino acid sequence of SEQ ID NO:1, a fragment thereof, or a variant thereof optionally comprising at least 50, 60, 70, 75, 80, 85, 90, 95, or more percent sequence identity to SEQ ID NO:1.

6. A chimeric fusion protein comprising an antigenic domain comprising the chimeric polypeptide of claim 1 and one or more additional domains.

7. The chimeric fusion protein of claim 6 wherein the one or more additional domains are selected from a signal peptide sequence, a trimerization domain, a cleavage site, a purification tag or report sequence, and one or more linker sequences.

8. The chimeric fusion protein of claim 6 comprising a trimerization domain, optionally wherein the trimerization domain is selected from GCN4-based isoleucine zipper (IZ), T4 bacteriophage fibritin foldon (Fd) trimerization domain, trimerization domain of collagen, the HIV gp41 trimerization domain, and the transmembrane domain and cytoplasmic tail of RSV or hMPV F protein, optionally wherein the trimerization domain comprises the amino acid sequence of any one of SEQ ID NOS:19-26, 44, or 45, and/or
a signal peptide sequence, optionally wherein the peptide signal sequence is derived from RSV F, optionally wherein the signal peptide sequence comprises the amino acid sequence of SEQ ID NO:5, and/or
a purification tag, optionally wherein the purification tag comprises six consecutive histidine residues.

9.-10. (canceled)

11. The chimeric fusion protein of claim 6, wherein when present, the orientation of the chimeric fusion protein domains is from N-terminus to C-terminus: signal peptide sequence-antigenic domain-trimerization domain-cleavage site-purification tag, optionally wherein one or more pairs of domains are separated by a linker sequence.

12. The chimeric fusion protein of claim 6 comprising the amino acid sequence of any one of SEQ ID NOS:2, or 41-43 or 46-47, or fragment, or variant thereof with at least 70% sequence identity thereto.

13. A nucleic acid encoding the chimeric polypeptide of claim 1.

14. The nucleic acid of claim 13, wherein the nucleic acid is mRNA.

15. A vector comprising the nucleic acid of claim 13 operably linked to an expression control sequence, optionally wherein the vector is a viral vector.

16. (canceled)

17. A recombinant virus genome or antigenome comprising the nucleic acid of claim 13, optionally wherein the recombinant virus is a recombinant RSV or recombinant hMPV, optionally wherein the recombinant virus is an attenuated virus.

18. A recombinant virus comprising the recombinant virus genome of claim 17.

19. A recombinant virus comprising the chimeric polypeptide of claim 1, optionally wherein the virus is replication competent or replication incompetent optionally wherein the recombinant virus is a recombinant RSV or recombinant hMPV, optionally wherein the recombinant virus is an attenuated virus.

20. (canceled)

21. A method of making an chimeric polypeptide or fusion protein comprising expressing a nucleic acid encoding the chimeric polypeptide of claim 1 in cells, optionally mammalian or insect cells, and isolating the expressed chimeric polypeptide or fusion protein, optionally wherein the expressed chimeric polypeptide or fusion protein is a multimeric protein such as a trimer.

22. A macromolecule comprising multimerizations of a mature form of the chimeric polypeptide of claim 1, optionally wherein the mature form of the chimeric polypeptide or fusion protein comprises cleavage of chimeric polypeptide or fusion protein.

23. (canceled)

24. A pharmaceutical composition comprising the chimeric polypeptide of claim 1, optionally further comprising an adjuvant.

25. (canceled)

26. A method of inducing or increasing an immune response in a subject in need thereof comprising administering the subject the pharmaceutical composition of claim 24.

27. The method of claim 26, wherein the pharmaceutical composition is administered in an effective amount to increase immunity against one or more pneumoviruses in the subject, optionally wherein the one or more pneumoviruses comprise RSV, hMPV, or both.

28. (canceled)

29. The method of claim 27, wherein increased immunity comprises increase neutralizing antibodies against the one or more pneumoviruses.

30. (canceled)

31. The method of claim 26, wherein the composition is administered prophylactically.

32. The method of claim 26, wherein administration reduces viral infection and/or one or more symptoms caused by viral infection in the subject.

33. The method of claim 26, wherein the subject is an infant, child, adult optionally elderly adult; optionally wherein the subject is a human.


SEQUENCE LISTING (47 sequences)

SEQ ID NO: 1 - protein, 494 aa, synthetic construct
HGLKESYLEESCSTITEGYLSVLRTGWYTSVITIELSNIKENKCNGTDAKVKLIKQELDKYKSAVTELQLLMQSTPATNNRARRELPRFMNYTLNNTKNTNVTLSKKRKRRSAIASGIAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQ

SEQ ID NO: 2 - protein, 576 aa, synthetic construct
MELPILKTNAITAILAAVTLCFASSHGLKESYLEESCSTITEGYLSVLRTGWYTSVITIELSNIKENKCNGTDAKVKLIKQELDKYKSAVTELQLLMQSTPATNNRARRELPRFMNYTLNNTKNTNVTLSKKRKRRSAIASGIAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGSGRMKQIEDKIEEILSKIYHIENEIARIKKLIGEAGSGRENLYFQGGSGGHHHHHH

SEQ ID NO: 3 - protein, 574 aa, Human Respiratory syncytial virus
MELPILKTNAITAILAAVTLCFASSQNITEEFYQTTCSAVSKGYLSALRTGWYTSVITIELSNIKENKCNGTDAKVKLIKQELDKYKSAVTELQLLMQSTPATNNRARRELPRFMNYTLNNTKNTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTINIMITTIIIVIIVILLSLIAVGLLLYCKARSTPVTLSKDQLSGINNIAFSN

SEQ ID NO: 4 - protein, 538 aa, Human metapneumovirus
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSKKRKRRVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGGGGGSGYIPEAPRDQAYVRKDGEWVLLSTFLGGTEGRHHHHHH

SEQ ID NO: 5 - protein, 25 aa, synthetic construct
MELPILKTNAITAILAAVTLCFASS

SEQ ID NO: 6 - protein, 16 aa, synthetic construct
MSWKVVIIFSLLITPQ

SEQ ID NO: 7 - protein, 4 aa, synthetic construct
RARR

SEQ ID NO: 8 - protein, 6 aa, synthetic construct
KKRKRR

SEQ ID NO: 9 - protein, 7 aa, synthetic construct
ENLYFQG

SEQ ID NO: 10 - skipped

SEQ ID NO: 11 - protein, 7 aa, synthetic construct
KRKRKKR

SEQ ID NO: 12 - protein, 4 aa, synthetic construct
RQSR

SEQ ID NO: 13 - skipped

SEQ ID NO: 14 - protein, 8 aa, synthetic construct
DXEXNPGP

SEQ ID NO: 15 - protein, 18 aa, synthetic construct
EGRGSLLTCGDVEENPGP

SEQ ID NO: 16 - protein, 19 aa, synthetic construct
ATNFSLLKQAGDVEENPGP

SEQ ID NO: 17 - protein, 20 aa, synthetic construct
QCTNYALLKLAGDVESNPGP

SEQ ID NO: 18 - protein, 22 aa, synthetic construct
VKQTLNFDLLKLAGDVESNPGP

SEQ ID NO: 19 - protein, 33 aa, synthetic construct
RMKQIEDKIEEILSKIYHIENEIARIKKLIGEA

SEQ ID NO: 20 - protein, 4 aa, synthetic construct
GSGG

SEQ ID NO: 21 - protein, 36 aa, synthetic construct
RMKQIEDKIEEILSKIYHIENEIARIKKLIGERGGR

SEQ ID NO: 22 - protein, 37 aa, synthetic construct
RMKQIEDKIEEILSKIYHIENEIARIKKLIGNRTGGR

SEQ ID NO: 23 - protein, 37 aa, synthetic construct
RMKQIEDKIENITSKIYHIENEIARIKKLIGNRTGGR

SEQ ID NO: 24 - protein, 37 aa, synthetic construct
RMKQIEDKIEEILSKIYNITNEIARIKKLIGNRTGGR

SEQ ID NO: 25 - protein, 37 aa, synthetic construct
RMKQIEDKIENITSKIYNITNEIARIKKLIGNRTGGR

SEQ ID NO: 26 - protein, 29 aa, synthetic construct
GSGYIPEAPRDGQAYVRKDGEWVLLSTFL

SEQ ID NO: 27 - protein, 4 aa, synthetic construct
GSGS

SEQ ID NO: 28 - protein, 4 aa, synthetic construct
GGGS

SEQ ID NO: 29 - protein, 5 aa, synthetic construct
GGSGG

SEQ ID NO: 30 - protein, 5 aa, synthetic construct
GGGGS

SEQ ID NO: 31 - protein, 10 aa, synthetic construct
GGGGSGGGGS

SEQ ID NO: 32 - protein, 20 aa, synthetic construct
GGGGSGGGGSGGGGSGGGGS

SEQ ID NO: 33 - protein, 15 aa, synthetic construct
GGGGSGGGGSGGGGS

SEQ ID NO: 34 - protein, 8 aa, synthetic construct
DYKDDDDK

SEQ ID NO: 35 - protein, 6 aa, synthetic construct
YPYDVP

SEQ ID NO: 36 - protein, 10 aa, synthetic construct
ILKKATAYIL

SEQ ID NO: 37 - protein, 10 aa, synthetic construct
EQKLISEEDL

SEQ ID NO: 38 - protein, 4 aa, synthetic construct
RRRR

SEQ ID NO: 39 - protein, 6 aa, synthetic construct
RRRRRR

SEQ ID NO: 40 - protein, 539 aa, Human metapneumovirus
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGFLIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGFIIVIILIAVLGSTMILVSVFIIIKKTKKPTGAPPELSGVTNNGFIPHN

SEQ ID NO: 41 - protein, 530 aa, synthetic construct
HGLKESYLEESCSTITEGYLSVLRTGWYTSVITIELSNIKENKCNGTDAKVKLIKQELDKYKSAVTELQLLMQSTPATNNRARRELPRFMNYTLNNTKNTNVTLSKKRKRRSAIASGIAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGSGRMKQIEDKIEEILSKIYHIENEIARIKKLIGEA

SEQ ID NO: 42 - protein, 555 aa, synthetic construct
MELPILKTNAITAILAAVTLCFASSHGLKESYLEESCSTITEGYLSVLRTGWYTSVITIELSNIKENKCNGTDAKVKLIKQELDKYKSAVTELQLLMQSTPATNNRARRELPRFMNYTLNNTKNTNVTLSKKRKRRSAIASGIAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGSGRMKQIEDKIEEILSKIYHIENEIARIKKLIGEA

SEQ ID NO: 43 - protein, 551 aa, synthetic construct
HGLKESYLEESCSTITEGYLSVLRTGWYTSVITIELSNIKENKCNGTDAKVKLIKQELDKYKSAVTELQLLMQSTPATNNRARRELPRFMNYTLNNTKNTNVTLSKKRKRRSAIASGIAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGSGRMKQIEDKIEEILSKIYHIENEIARIKKLIGEAGSGRENLYFQGGSGGHHHHHH

SEQ ID NO: 44 - protein, 50 aa, synthetic construct
IMITTIIIVIIVILLSLIAVGLLLYCKARSTPVTLSKDQLSGINNIAFSN

SEQ ID NO: 45 - protein, 49 aa, synthetic construct
FIIVIILIAVLGSTMILVSVFIIIKKTKKPTGAPPELSGVTNNGFIPHN

SEQ ID NO: 46 - protein, 540 aa, synthetic construct
HGLKESYLEESCSTITEGYLSVLRTGWYTSVITIELSNIKENKCNGTDAKVKLIKQELDKYKSAVTELQLLMQSTPATNNRARRELPRFMNYTLNNTKNTNVTLSKKRKRRSAIASGIAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGSGRMKQIEDKIEEILSKIYHIENEIARIKKLIGEAGSGRENLYFQ

SEQ ID NO: 47 - protein, 565 aa, synthetic construct
MELPILKTNAITAILAAVTLCFASSHGLKESYLEESCSTITEGYLSVLRTGWYTSVITIELSNIKENKCNGTDAKVKLIKQELDKYKSAVTELQLLMQSTPATNNRARRELPRFMNYTLNNTKNTNVTLSKKRKRRSAIASGIAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTSGRENLYFQGSGRMKQIEDKIEEILSKIYHIENEIARIKKLIGEAGSGRENLYFQ